(19) United States
(12) Patent Application Publication
(10) Pub. No.: US 2026/0238478 A1
(43) Pub. Date: Aug. 13, 2026

(54) ENCRYPTION CONNECTION SYSTEM, METHOD, AND PORTABLE COLD THERAPY DEVICE

(71) Applicants: Quanqin DAI, Irvine, CA (US); Pu JIANG, Shenzhen (CN)

(72) Inventors: Quanqin DAI, Irvine, CA (US); Pu JIANG, Shenzhen (CN)

(21) Appl. No.: 19/460,664

(22) Filed: Jan. 27, 2026

Related U.S. Application Data
(63) Continuation-in-part of application No. 19/098,892, filed on Apr. 2, 2025.
Application No. 19/098,892 is a continuation-in-part of application No. 18/766,559, filed on Jul. 8, 2024, now Pat. No. 12,290,469.
Application No. 18/766,559 is a continuation-in-part of application No. 18/386,568, filed on Nov. 2, 2023, now Pat. No. 12,070,413.

(30) Foreign Application Priority Data
Sep. 8, 2023 (CN) 202322449198.9

(51) Int. Cl. H04L 9/32; A61F 7/00; G16H 40/67; A61F 7/02

(52) U.S. Cl. CPC H04L 9/32; A61F 7/0085; G16H 40/67; A61F 2007/0056; A61F 2007/0077; A61F 2007/0093; A61F 2007/0094; A61F 2007/0225

(57) ABSTRACT

An encryption connection system is disclosed, comprising an accessory module, a processing module, and a host module, and wherein the host module controls an operating state of the cold therapy system according to a result of the verification and matching. The accessory module stores the identity information. The host module obtains this identity information through the processing module and performs matching verification. The cold therapy system is only authorized to start treatment after the verification is passed, it can accurately identify authorized body wraps, thereby effectively preventing the connection of unauthorized body wraps and ensuring the exclusivity and safety of the treatment. This encryption connection system fundamentally addresses issues such as medical operation risks, commercial infringement hazards, and data interference caused by the lack of a security authentication mechanism in traditional cold therapy devices, thereby enhancing the overall system's reliability and compliance.

### CROSS REFERENCE TO RELATED APPLICATIONS

This application is a continuation-in-part of U.S. Application No. 19/098,892, filed on April 2, 2025, which is a continuation-in-part of U.S. Application No. 18/766,559, filed on July 8, 2024, which is a continuation-in-part of U.S. Application No. 18/386,568, filed on November 2, 2023, which claims the benefit of priority to Chinese Application No. 202322449198.9, filed on September 8, 2023, all of which are hereby incorporated by reference in their entireties.

### TECHNICAL FIELD

The various embodiments described in this document relate in general to the field of medical devices, and more particularly, to an encryption connection system, method, and portable cold therapy device.

### BACKGROUND

The cold therapy system and device (e.g., body wraps) are widely used in medical fields such as exercise rehabilitation, pain relief, swelling elimination, wound healing and the like. By circulating the cooled liquid in the cold therapy system into the body wrap, the cold compress to be treated parts of the human body can be realized.

Currently, cold therapy devices generally lack an identity authentication or encrypted communication mechanism between the cold therapy system and the body wrap. This allows unauthorized body wraps to be connected and used arbitrarily, leading to the following issues:

First, there are safety risks. Unauthorized body wraps may fail to meet material standards or have mismatched thermal conduction parameters, which can lead to loss of temperature control. This not only affects therapeutic efficacy but may also cause safety incidents such as frostbite.

Second, commercial erosion occurs. The arbitrary substitution of original equipment manufacturer (OEM) body wraps with third-party ones directly impacts the OEM consumables market, harming the enterprise's sustainable investment in research and development and its commercial ecosystem.

Third, data distortion and contamination arise. Non-standard body wraps introduce abnormal treatment parameters, causing distortion in data collection. The intermingling of usage records contaminates the OEM data system, severely compromising the credibility of data-based therapeutic efficacy evaluation, health analysis, and device optimization efforts.

### SUMMARY

With respect to the technical problems of medical risks, commercial infringement, and data interference existing in conventional cold therapy devices, embodiments of the present disclosure aim to provide an encryption connection system, which can fundamentally address issues such as medical safety risks, commercial infringement, and data interference caused by the lack of secure authentication between the cold therapy system and the body wrap in existing cold therapy devices.

According to a first aspect, embodiments of the present disclosure provide an encryption connection system, comprising: an accessory module, disposed on a body wrap and configured to store an identity information; a processing module, connected to the accessory module and configured to obtain the identity information; and a host module, disposed in a cold therapy system and connected to the processing module, configured to obtain the identity information through the processing module and perform verification and matching; and wherein the host module controls an operating state of the cold therapy system according to a result of the verification and matching.

In accordance with the encryption connection system provided in the present disclosure, the advantageous effects include the following. The encryption connection system establishes a complete encrypted connection mechanism between the cold therapy system and the body wrap. Through the coordinated operation of the accessory module, the processing module, and the host module, secure authentication based on identity information is achieved. Specifically, the accessory module stores the identity information. The host module obtains this identity information through the processing module and performs matching verification. The cold therapy system is only authorized to start treatment after the verification is passed. This design can accurately identify authorized body wraps, thereby effectively preventing the connection of unauthorized body wraps and ensuring the exclusivity and safety of the treatment. This encryption connection system fundamentally addresses issues such as medical operation risks, commercial infringement hazards, and data interference caused by the lack of a security authentication mechanism in traditional cold therapy devices, thereby enhancing the overall system's reliability and compliance.

According to a second aspect, embodiments of the present disclosure provide an encryption connection method, comprising: initiating an identity authentication request from a host module of a cold therapy system to a processing module; the processing module, according to the identity authentication request, acquiring identity information stored in an accessory module of a body wrap; the host module receiving the identity information returned by the processing module, and performing verification and matching on the identity information based on a preset verification rule; the host module generating a control instruction according to a verification and matching result to control an operating state of the cold therapy system; wherein, when the verification and matching is successful, the host module authorizes the cold therapy system to start operation; when the verification and matching fails, the host module maintains the cold therapy system in a locked or off state.

In accordance with the encryption connection method provided in the present disclosure, the advantageous effects include the following. The encryption connection method provides a hierarchical, clear, secure, and reliable authentication control process. This method involves the host module actively initiating an authentication request, driving the processing module to acquire identity information from the accessory module, and then having the host module complete verification and matching based on preset rules. Finally, the operating state of the cold therapy system is determined according to the matching result. This process transforms the hardware authentication mechanism into standardized software logic steps, ensuring that only authorized body wraps that pass strict verification can trigger the treatment procedure, thereby achieving security control over the medical process at the operational level. The method is rigorous in process, clear in judgment, and easy to integrate into existing cold therapy device control systems. It can effectively prevent unauthorized use and ensure the compliance and controllability of treatment.

According to a third aspect, embodiments of the present disclosure provide a portable cold therapy device, comprising: a cold therapy system; a body wrap; and an encryption connection system, the encryption connection system comprising: an accessory module, disposed on the body wrap and configured to store an identity information; a processing module, connected to the accessory module and configured to acquire the identity information; and a host module, disposed in the cold therapy system and connected to the processing module, configured to acquire the identity information through the processing module and perform verification and matching; and wherein the host module controls an operating state of the cold therapy system according to a result of the verification and matching.

In accordance with the portable cold therapy device provided in the present disclosure, the advantageous effects include the following. The portable cold therapy device integrates the encryption connection system as a core security component deeply between the cold therapy system and the body wrap of the portable cold therapy device, achieving device-level integrated security protection. Through the coordination of the built-in accessory module, processing module, and host module, the device completes a full hardware-level closed loop from identity identification and verification to operation control, ensuring that the device only functions normally when paired with an authorized and legitimate body wrap. This deeply integrated design fundamentally eliminates the possibility of using unauthorized body wraps, enhancing the product's own safety and reliability while also strengthening the exclusivity of its business model.

In some examples, embodiments of the present disclosure provide a portable system for cold therapy with optional heat and compression therapy, applied to a body wrap, comprising a cooling mechanism, a heating mechanism, a reservoir, and a control assembly; the cooling mechanism comprises a cooling component and a first heat exchanger, and the cooling component is used for cooling the first heat exchanger; the heating mechanism comprises a heating component and a second heat exchanger, and the heating component is used for heating the second heat exchanger; the control assembly is electrically connected to the cooling component and the heating component, respectively; the cooling component and the heating component are placed outside the reservoir, respectively; and wherein: the reservoir comprises a liquid outlet of the reservoir; the first heat exchanger comprises a liquid inlet of the first heat exchanger, and a liquid outlet of the first heat exchanger; the second heat exchanger comprises a liquid inlet of the second heat exchanger, and a liquid outlet of the second heat exchanger; the liquid in the reservoir can flow into the first heat exchanger through the liquid outlet of the reservoir and the liquid inlet of the first heat exchanger, or flow into the second heat exchanger through the liquid outlet of the reservoir and the liquid inlet of the second heat exchanger.

When the liquid in the reservoir flows into the first heat exchanger first, and then flows into the body wrap through the liquid outlet of the first heat exchanger and the liquid inlet of the body wrap; the liquid in the body wrap can flow into the first heat exchanger first through the liquid outlet of the body wrap and the liquid inlet of the first heat exchanger, and then flow into the body wrap again, or the liquid in the body wrap can flow back to the reservoir through the liquid outlet of the body wrap and the liquid outlet of the reservoir.

When the liquid in the reservoir flows into the second heat exchanger first, and then flows into the body wrap through the liquid outlet of the second heat exchanger and the liquid inlet of the body wrap; the liquid in the body wrap can flow into the second heat exchanger first through the liquid outlet of the body wrap and the liquid inlet of the second heat exchanger, and then flow into the body wrap again, or the liquid in the body wrap can flow back to the reservoir through the liquid outlet of the body wrap and the liquid outlet of the reservoir.

In accordance with the portable system for cold therapy with optional heat and compression therapy provided in the present disclosure, the advantageous effects include the following. In some examples, by setting the cooling component and the first heat exchanger, the first heat exchanger can be cooled through the cooling component, the liquid in the reservoir can flow into the body wrap after cooling through the first heat exchanger, the liquid in the body wrap can flow into the first heat exchanger again for cooling, or the liquid in the body wrap can flow back into the reservoir, providing the body wrap with a cold compression function, and the remaining liquid in the reservoir will no longer participate in the circulation, to greatly improve the cooling efficiency, and reduce the power consumption. Under the condition of the ambient temperature of 25-28℃, with an area of 1m² of the body wrap, a cooling rate is such that the surface temperature of the body wrap reaches 5℃ within about 3 minutes, a cooling power is about 75 watts, and the achieved cooling capacity is about 250 watts. The device exhibits excellent cooling effects, high efficiency, and stability. In some examples, by setting the heating component and the second heat exchanger, the second heat exchanger can be heated through the heating component, the liquid in the reservoir can flow into the body wrap after heating through the second heat exchanger, the liquid in the body wrap can flow into the second heat exchanger again for heating, or the liquid in the body wrap can flow back into the reservoir, providing the body wrap with a hot compression function, and the remaining liquid in the reservoir will no longer participate in the circulation, to greatly improve the heating efficiency, and reduce the power consumption. Under the condition of the ambient temperature of 25-28℃, with an area of 1m² of the body wrap, a heating rate is such that the surface temperature of the body wrap reaches 43℃ within about 5 minutes, a heating power is about 150 watts, and the achieved heating capacity is about 150 watts. The device exhibits excellent heating effects, high efficiency, and stability. The cooling and heating mechanisms are simultaneously incorporated into the portable device for cold therapy with optional heat and compression therapy, provided in the present disclosure. The device has a compact, small, and lightweight structure, facilitating convenient portability.

Furthermore, the heat exchanger of the cooling mechanism and the heating mechanism are set separately, so that the first heat exchanger allows pre-cooling, and the second heat exchanger allows pre-heating, thereby enabling immediate use when the user requires therapy.

In some examples, embodiments of the present disclosure provide a portable device for cold therapy with optional heat and compression therapy, including the portable system for cold therapy with optional heat and compression therapy and a body wrap; wherein the portable system for cold therapy with optional heat and compression therapy comprises a cooling mechanism, a heating mechanism, a reservoir, and a control assembly; the cooling mechanism comprises a cooling component and a first heat exchanger, and the cooling component is used for cooling the first heat exchanger; the heating mechanism comprises a heating component and a second heat exchanger, and the heating component is used for heating the second heat exchanger; the control assembly is electrically connected to the cooling component and the heating component, respectively; the cooling component and the heating component are placed outside the reservoir, respectively; and wherein: the reservoir comprises a liquid outlet of the reservoir; the first heat exchanger comprises a liquid inlet of the first heat exchanger, and a liquid outlet of the first heat exchanger; the second heat exchanger comprises a liquid inlet of the second heat exchanger, and a liquid outlet of the second heat exchanger; the liquid in the reservoir can flow into the first heat exchanger through the liquid outlet of the reservoir and the liquid inlet of the first heat exchanger, or flow into the second heat exchanger through the liquid outlet of the reservoir and the liquid inlet of the second heat exchanger.

When the liquid in the reservoir flows into the first heat exchanger first, and then flows into the body wrap through the liquid outlet of the first heat exchanger and the liquid inlet of the body wrap; the liquid in the body wrap can flow into the first heat exchanger first through the liquid outlet of the body wrap and the liquid inlet of the first heat exchanger, and then flow into the body wrap again, or the liquid in the body wrap can flow back to the reservoir through the liquid outlet of the body wrap and the liquid outlet of the reservoir.

When the liquid in the reservoir flows into the second heat exchanger first, and then flows into the body wrap through the liquid outlet of the second heat exchanger and the liquid inlet of the body wrap; the liquid in the body wrap can flow into the second heat exchanger first through the liquid outlet of the body wrap and the liquid inlet of the second heat exchanger, and then flow into the body wrap again, or the liquid in the body wrap can flow back to the reservoir through the liquid outlet of the body wrap and the liquid outlet of the reservoir.

In accordance with the portable device for cold therapy with optional heat and compression therapy provided in the present disclosure, the advantageous effects include the following. In some examples, by setting the cooling component and the first heat exchanger, the first heat exchanger can be cooled through the cooling component, the liquid in the reservoir can flow into the body wrap after cooling through the first heat exchanger, the liquid in the body wrap can flow into the first heat exchanger again for cooling, or the liquid in the body wrap can flow back into the reservoir, providing the body wrap with a cold compression function, and the remaining liquid in the reservoir will no longer participate in the circulation, to greatly improve the cooling efficiency, and reduce the power consumption. Under the condition of the ambient temperature of 25-28℃, with an area of 1m² of the body wrap, a cooling rate is such that the surface temperature of the body wrap reaches 5℃ within about 3 minutes, a cooling power is about 75 watts, and the achieved cooling capacity is about 250 watts. The device exhibits excellent cooling effects, high efficiency, and stability. In some examples, by setting the heating component and the second heat exchanger, the second heat exchanger can be heated through the heating component, the liquid in the reservoir can flow into the body wrap after heating through the second heat exchanger, the liquid in the body wrap can flow into the second heat exchanger again for heating, or the liquid in the body wrap can flow back into the reservoir, providing the body wrap with a hot compression function, and the remaining liquid in the reservoir will no longer participate in the circulation, to greatly improve the heating efficiency, and reduce the power consumption. Under the condition of the ambient temperature of 25-28℃, with an area of 1m² of the body wrap, a heating rate is such that the surface temperature of the body wrap reaches 43℃ within about 5 minutes, a heating power is about 150 watts, and the achieved heating capacity is about 150 watts. The device exhibits excellent heating effects, high efficiency, and stability. The cooling and heating mechanisms are simultaneously incorporated into the portable device for cold therapy with optional heat and compression therapy, provided in the present disclosure. The device has a compact, small, and lightweight structure, facilitating convenient portability.

Furthermore, the heat exchanger of the cooling mechanism and the heating mechanism are set separately, so that the first heat exchanger allows pre-cooling, and the second heat exchanger allows pre-heating, thereby enabling immediate use when the user requires therapy.

In some examples, embodiments of the present disclosure provide a portable system for cold therapy with optional compression therapy, applied to a body wrap, comprising a cooling mechanism, a reservoir, and a control assembly; wherein the cooling mechanism comprises a compressor, a condenser, a fan, and a heat exchanger, and the control assembly is electrically connected to the compressor, and the fan, respectively; and wherein: the compressor, the condenser, the fan, and the heat exchanger are placed outside the reservoir, respectively; the compressor has an air inlet connected to the heat exchanger through a first tube; the heat exchanger is connected to the condenser through a second tube; the condenser is connected to an air outlet of the compressor through a third tube; the first tube and the second tube are connected by fluid, and the second tube and the third tube are connected by fluid; the fan has a working surface facing the condenser; and wherein, the reservoir comprises a liquid outlet of the reservoir; the heat exchanger comprises an air inlet of the heat exchanger, an air outlet of the heat exchanger, a liquid inlet of the heat exchanger, and a liquid outlet of the heat exchanger; wherein the air outlet of the heat exchanger is connected to the first tube, the air inlet of the heat exchanger is connected to the second tube, the liquid inlet of the heat exchanger is connected to the liquid outlet of the reservoir, the liquid outlet of the heat exchanger is connected to a liquid inlet of the body wrap, and a liquid outlet of the body wrap is connected to the liquid outlet of the reservoir and the liquid inlet of the heat exchanger.

Wherein the liquid in the reservoir flows into the heat exchanger first through the liquid outlet of the reservoir and the liquid inlet of the heat exchanger, and then flows into the body wrap through the liquid outlet of the heat exchanger and the liquid inlet of the body wrap; the liquid in the body wrap can flow into the heat exchanger first through the liquid outlet of the body wrap and the liquid inlet of the heat exchanger, and then flow into the body wrap again, or the liquid in the body wrap can flow back to the reservoir through the liquid outlet of the body wrap and the liquid outlet of the reservoir.

In accordance with the portable system for cold therapy with optional compression therapy provided in the present disclosure, the advantageous effects include the following. The cooling mechanism is equipped with the compressor, the condenser, the fan, and the heat exchanger. High temperature and high-pressure refrigerant gas is transported from the compressor to the condenser through the third tube, at this time, the temperature of the refrigerant gas is high, and the fan is utilized to cool the refrigerant gas in the condenser, and the refrigerant gas is liquefied. Refrigerant liquid is transported from the condenser to the heat exchanger through the second tube. In the heat exchanger, the refrigerant liquid undergoes heat exchange with the liquid in the heat exchanger, absorbs heat from the liquid and gasifies, and the liquid temperature decreases. Refrigerant gas is transported from the heat exchanger to the compressor through the first tube, and the compressor compresses the refrigerant gas. The heat exchanger is arranged on the outside of the reservoir, the liquid in the reservoir flows into the heat exchanger for cooling through the liquid outlet of the reservoir and then flows into the body wrap. The liquid in the body wrap can flow into the heat exchanger for cooling again, or the liquid in the body wrap can flow back into the reservoir, providing the body wrap with a cold compression function, and the remaining liquid in the reservoir will no longer participate in the circulation, to greatly improve the cooling efficiency, and reduce the power consumption. Under the condition of the ambient temperature of 25-28℃, with an area of 1m² of the body wrap, a cooling rate is such that the surface temperature of the body wrap reaches 5℃ within about 5 minutes, a cooling power is about 75 watts, and the achieved cooling capacity is about 250 watts. The system exhibits excellent cooling effects, high efficiency, stability, and low power consumption. The device has a compact, small, and lightweight structure, facilitating convenient portability.

In some examples, embodiments of the present disclosure provide a portable device for cold therapy with optional compression therapy, including the portable system for cold therapy with optional compression therapy and a body wrap; wherein the portable system for cold therapy with optional compression therapy comprises a cooling mechanism, a reservoir, and a control assembly; wherein the cooling mechanism comprises a compressor, a condenser, a fan, and a heat exchanger, and the control assembly is electrically connected to the compressor, and the fan, respectively; and wherein: the compressor, the condenser, the fan, and the heat exchanger are placed outside the reservoir, respectively; the compressor has an air inlet connected to the heat exchanger through a first tube; the heat exchanger is connected to the condenser through a second tube; the condenser is connected to an air outlet of the compressor through a third tube; the first tube and the second tube are connected by fluid, and the second tube and the third tube are connected by fluid; the fan has a working surface facing the condenser; and wherein, the reservoir comprises a liquid outlet of the reservoir; the heat exchanger comprises an air inlet of the heat exchanger, an air outlet of the heat exchanger, a liquid inlet of the heat exchanger, and a liquid outlet of the heat exchanger; wherein the air outlet of the heat exchanger is connected to the first tube, the air inlet of the heat exchanger is connected to the second tube, the liquid inlet of the heat exchanger is connected to the liquid outlet of the reservoir, the liquid outlet of the heat exchanger is connected to a liquid inlet of the body wrap, and a liquid outlet of the body wrap is connected to the liquid outlet of the reservoir and the liquid inlet of the heat exchanger.

Wherein the liquid in the reservoir flows into the heat exchanger first through the liquid outlet of the reservoir and the liquid inlet of the heat exchanger, and then flows into the body wrap through the liquid outlet of the heat exchanger and the liquid inlet of the body wrap; the liquid in the body wrap can flow into the heat exchanger first through the liquid outlet of the body wrap and the liquid inlet of the heat exchanger, and then flow into the body wrap again, or the liquid in the body wrap can flow back to the reservoir through the liquid outlet of the body wrap and the liquid outlet of the reservoir.

In accordance with the portable device for cold therapy with optional compression therapy provided in the present disclosure, the advantageous effects include the following. In some examples, the cooling mechanism is equipped with the compressor, the condenser, the fan, and the heat exchanger. High temperature and high-pressure refrigerant gas is transported from the compressor to the condenser through the third tube, at this time, the temperature of the refrigerant gas is high, and the fan is utilized to cool the refrigerant gas in the condenser, and the refrigerant gas is liquefied. Refrigerant liquid is transported from the condenser to the heat exchanger through the second tube. In the heat exchanger, the refrigerant liquid undergoes heat exchange with the liquid in the heat exchanger, absorbs heat from the liquid and gasifies, and the liquid temperature decreases. Refrigerant gas is transported from the heat exchanger to the compressor through the first tube, and the compressor compresses the refrigerant gas. The heat exchanger is arranged on the outside of the reservoir, the liquid in the reservoir flows into the heat exchanger for cooling through the liquid outlet of the reservoir and then flows into the body wrap. The liquid in the body wrap can flow into the heat exchanger for cooling again, or the liquid in the body wrap can flow back into the reservoir, providing the body wrap with a cold compression function, and the remaining liquid in the reservoir will no longer participate in the circulation, to greatly improve the cooling efficiency, and reduce the power consumption. Under the condition of the ambient temperature of 25-28℃, with an area of 1m² of the body wrap, a cooling rate is such that the surface temperature of the body wrap reaches 5℃ within about 5 minutes, a cooling power is about 75 watts, and the achieved cooling capacity is about 250 watts. The system exhibits excellent cooling effects, high efficiency, stability, and low power consumption. In some examples, the air pumps and the solenoid valves are provided to inflate the body wrap, allowing the body wrap to conform more closely to therapy areas. This enhances the therapy effect of the cold therapy with optional compression therapy. Additionally, the combination of inflation and deflation allows the body wrap to provide medical effects such as massage and compression. The device has a compact, small, and lightweight structure, facilitating convenient portability. The device is versatile, offering capabilities for cold therapy, and/or massage, enhancing the user experience.

In some examples, embodiments of the present disclosure provide a portable system for cold therapy with optional heat and compression therapy, applied to a first body wrap, including a body wrap cooling mechanism, a reservoir, and a control assembly; the body wrap cooling mechanism includes a compressor, a condenser, a fan, and a heat exchanger; the control assembly is electrically connected to the compressor, and the fan, respectively; the compressor is placed outside the reservoir, and the condenser is placed adjacent to the reservoir and the fan, respectively; the heat exchanger is placed inside or underneath the reservoir, and is in direct or indirect contact with liquid in the reservoir to cool the liquid; the compressor has an air outlet connected to the heat exchanger through a first tube; the heat exchanger is connected to the condenser through a second tube; the condenser is connected to an air inlet of the compressor through a third tube; the first tube is communicated with the second tube, and the second tube is communicated with the third tube; the fan has a working surface facing the condenser; and the reservoir includes a liquid outlet of the reservoir and a liquid inlet of the reservoir, the liquid in the reservoir flows into the first body wrap through the liquid outlet of the reservoir, and liquid in the first body wrap flows into the reservoir through the liquid inlet of the reservoir.

In accordance with the portable system for cold therapy with optional heat and compression therapy provided in the present disclosure, the advantageous effects include the following. The body wrap cooling mechanism is equipped with the compressor, the condenser, the fan, and the heat exchanger. Liquid gas is transported from the compressor to the heat exchanger through the first tube. In the heat exchanger, the liquid gas undergoes heat exchange with the liquid in the reservoir, absorbs heat from the liquid and gasifies, and the liquid temperature in the reservoir decreases. The gasified gas is then transported from the heat exchanger to the condenser through the second tube. At this point, the temperature of the gasified gas transported from the heat exchanger to the condenser is relatively high, and the fan is utilized to cool the gasified gas in the condenser. The cooled gas is then transported from the condenser to the compressor through the third tube, and the compressor compresses and liquefies the gas. This process circulates to cool the liquid in the reservoir. A cooling rate is such that the liquid in the reservoir decreases from room temperature to 5°C within about 10 minutes. The cooling power is about 75 watts, and the achieved cooling capacity is about 150 watts. The system exhibits excellent cooling effects, high efficiency, stability, and low power consumption. The cooled liquid in the reservoir flows into the first body wrap through the liquid outlet of the reservoir. The liquid in the first body wrap flows into the reservoir through the liquid inlet of the reservoir, achieving a circulation of liquid and temperature reduction for cold therapy. The first body wrap is used for cold compression therapy on the body to achieve therapeutic effects. Additionally, an overall volume of the system is approximately 0.014 m³, with a mass of about 3.8 kg, which has a compact, small, and lightweight structure, facilitating convenient portability.

In some embodiments, the heat exchanger includes a first cooling plate, and the first cooling plate is placed inside the reservoir; the first cooling plate is provided with a first air inlet and a first air outlet; and the first air inlet is connected to the first tube, and the first air outlet is connected to the second tube.

In some embodiments, the first cooling plate is further provided with an optional liquid inlet of the first cooling plate and an optional liquid outlet of the first cooling plate, and the optional liquid inlet of the first cooling plate is connected to the liquid inlet of the reservoir through a tube.

In some embodiments, the heat exchanger includes a spiral tube, and the spiral tube is placed inside the reservoir; and the spiral tube has one end connected to the first tube and the other end connected to the second tube.

In some embodiments, the heat exchanger includes a second cooling plate, and the second cooling plate is placed underneath the reservoir; the second cooling plate is provided with a second air inlet and a second air outlet, the second air inlet is connected to the first tube, and the second air outlet is connected to the second tube; and the reservoir includes a heat-conducting material.

In some embodiments, the reservoir is further provided with a liquid level detector and a temperature sensor inside, and the liquid level detector and the temperature sensor are both electrically connected to the control assembly.

In some embodiments, the second tube includes a capillary tube section, and the capillary tube section is placed between the heat exchanger and the condenser.

In some embodiments, the condenser includes a heat conduction tube, and the heat conduction tube is U-shaped and is bent along an inner wall of the condenser; and the heat conduction tube has one end extending out of the condenser to be connected to the capillary tube section and the other end extending out of the condenser to be connected to the compressor.

In some embodiments, the system further includes a water pump, the liquid outlet of the reservoir is connected to a liquid inlet of the first body wrap through the water pump, and the water pump is electrically connected to the control assembly; in response to the water pump working, the liquid in the reservoir flows into the first body wrap through the liquid outlet of the reservoir and the liquid inlet of the first body wrap, and the liquid in the first body wrap flows back into the reservoir through a liquid outlet of the body wrap and the liquid inlet of the reservoir.

In some embodiments, the system further includes a four-way valve, the compressor is connected to the four-way valve and then to the heat exchanger through the first tube, and the condenser is connected to the four-way valve and then to the compressor through the third tube; the four-way valve is electrically connected to the control assembly, and the four-way valve is controlled by the control assembly to change a gas flow direction, enabling air in the compressor to pass through the condenser first and then through the heat exchanger.

In some embodiments, the system further includes a body wrap heating mechanism, the body wrap heating mechanism includes a heating tube; and the heating tube is placed inside the reservoir and is electrically connected to the control assembly.

In some embodiments, the system further includes a first air pump, a first solenoid valve, and a first air tube; the first air pump is connected to the first air tube through the first solenoid valve, and the first air tube is connected to the first body wrap; the first air pump and the first solenoid valve are both electrically connected to the control assembly; and the first body wrap is inflated by the first air pump, and the first body wrap is deflated by the first solenoid valve.

In some embodiments, the system further includes a second air pump, a second solenoid valve, a third solenoid valve, a second air tube, and a third air tube; the second air pump is connected to the second air tube through the second solenoid valve, the second air pump is connected to the third air tube through the third solenoid valve, the second air tube is connected to a second body wrap, and the third air tube is connected to a third body wrap; the second air pump, the second solenoid valve, and the third solenoid valve are all electrically connected to the control assembly; and the second body wrap and the third body wrap are inflated by the second air pump, the second body wrap is deflated by the second solenoid valve, and the third body wrap is deflated by the third solenoid valve.

In some embodiments, the system further includes a housing, a bracket, and a connector, and the housing includes a bottom shell, an upper shell and a surface cover; the bracket is embedded in the housing, and the reservoir is placed on the bracket; an avoidance portion is provided on the bracket, and the condenser is embedded in the avoidance portion; an exterior side of the bottom shell is provided with a first groove, and the connector has one end inserted into the first groove and respectively connected to the liquid outlet of reservoir, the liquid inlet of the reservoir and the first air tube, and has the other end configured to be connected to a plurality of tubes, enabling the liquid outlet of the reservoir, the liquid inlet of the reservoir and the first air tube to be respectively connected to the first body wrap; the bottom shell is further provided with a first air hole and a second air hole, the first air hole and the second air hole are placed below the first groove; and one side of the first air hole is connected to the second air tube, one side of the second air hole is connected to the third air tube, and the other side of the first air hole and the other side of the second air hole are configured to be connected to tubes, enabling the second air tube to be connected to the second body wrap and the third air tube to be connected to the third body wrap.

In some embodiments, the control assembly further includes a circuit board, a display screen and a battery; the display screen is electrically connected to the circuit board; the display screen is placed on the surface cover; and an exterior side of the bottom shell is recessed towards the bracket to form a second groove, and the battery is placed in the second groove, and the battery includes a storage battery.

In some examples, embodiments of the present disclosure provide a portable device for cold therapy with optional heat and compression therapy, including the portable system for cold therapy with optional heat and compression therapy as described in any one of the embodiments as described above. The portable device for cold therapy with optional heat and compression therapy includes a first body wrap, and the first body wrap is connected to the reservoir through the liquid outlet of the reservoir and the liquid inlet of the reservoir, respectively.

In accordance with the portable device for cold therapy with optional heat and compression therapy provided in the present disclosure, the advantageous effects include the following. In some examples, the body wrap cooling mechanism is equipped with the compressor, the condenser, the fan, and the heat exchanger. Liquid gas is transported from the compressor to the heat exchanger through the first tube. In the heat exchanger, the liquid gas undergoes heat exchange with the liquid in the reservoir, absorbs heat from the liquid and gasifies, and the liquid temperature decreases. The gasified gas is then transported from the heat exchanger to the condenser through the second tube. At this point, the temperature of the gasified gas transported from the heat exchanger to the condenser is relatively high, and the fan is utilized to cool the gasified gas in the condenser. The cooled gas is then transported from the condenser to the compressor through the third tube, and the compressor compresses and liquefies the gas. This process circulates to cool the liquid in the reservoir. A cooling rate is such that the liquid decreases from room temperature to 5°C within about 10 minutes. A cooling power is about 75 watts, and the achieved cooling capacity is about 150 watts. The system exhibits excellent cooling effects, high efficiency, stability, and low power consumption. The liquid with decreased temperature flows into the body wrap through the liquid inlet of the body wrap, providing the body wrap with a cold compression function. In some examples, the air pumps and the solenoid valves are provided to inflate the body wrap, allowing the body wrap to conform more closely to therapy areas. This enhances the therapy effect of the cold therapy with optional heat and compression therapy. Additionally, the combination of inflation and deflation allows the body wrap to provide medical effects such as massage and compression. In some examples, the compressor, condenser, and fan are paused by the control assembly. The heating tube is activated by the control assembly to increase the temperature of the liquid in the reservoir. A heating rate is such that the liquid reaches 43°C from room temperature within about 6 minutes. The heating power is about 150 watts, and the achieved heating capacity is about 150 watts. The device exhibits excellent heating effects, high efficiency, and stability. The liquid with increased temperature flows into the body wrap through the liquid inlet of the body wrap, providing the body wrap with a hot compression function. The cooling and heating mechanisms are simultaneously incorporated into the portable device for cold therapy with optional heat and compression therapy, provided in the present disclosure. The overall volume of the device is approximately 0.014 m³, with a mass of about 3.8 kg, which has a compact, small, and lightweight structure, facilitating convenient portability. The device is versatile, offering capabilities for cold compression, hot compression, and/or massage, enhancing the user experience.

Furthermore, as the cooling and heating mechanisms in the present disclosure is placed inside or underneath the reservoir, which are able to directly or indirectly contact the liquid in the reservoir. This allows pre-cooling or pre-heating of the liquid, thereby enabling immediate use when the user requires therapy.

### BRIEF DESCRIPTION OF THE DRAWINGS

In order to more clearly illustrate the embodiments of the present disclosure or conventional technical solutions, accompanying drawings required in the embodiments or conventional technical solutions are briefly described below. It is apparent that the accompany drawings in the following description are merely some embodiments of the present disclosure. For a person of ordinary skills in the art, other drawings may also be obtained according to these drawings.

FIG. 1 is an exploded schematic structural diagram of a portable system for cold therapy with optional heat and compression therapy according to an embodiment of the present disclosure.

FIG. 2 is a first partial schematic structural diagram of a portable device for cold therapy with optional heat and compression therapy according to an embodiment of the present disclosure.

FIG. 3 is a second schematic structural diagram of a portable device for cold therapy with optional heat and compression therapy according to an embodiment of the present disclosure.

FIG. 4 is a third schematic structural diagram of a portable device for cold therapy with optional heat and compression therapy according to an embodiment of the present disclosure.

FIG. 5 is a first partial schematic structural diagram of a portable system for cold therapy with optional heat and compression therapy according to an embodiment of the present disclosure.

FIG. 6 is a second schematic structural diagram of a portable system for cold therapy with optional heat and compression therapy according to an embodiment of the present disclosure.

FIG. 7 is a third schematic structural diagram of a portable system for cold therapy with optional heat and compression therapy according to an embodiment of the present disclosure.

FIG. 8 is a fourth schematic structural diagram of a portable system for cold therapy with optional heat and compression therapy according to an embodiment of the present disclosure.

FIG. 9 is a fifth schematic structural diagram of a portable system for cold therapy with optional heat and compression therapy according to an embodiment of the present disclosure.

FIG. 10 is a sixth schematic structural diagram of a portable system for cold therapy with optional heat and compression therapy according to an embodiment of the present disclosure.

FIG. 11 is a first partial schematic structural diagram of a portable system for cold therapy with optional heat and compression therapy in a manner of a heat exchanger being a first cooling plate.

FIG. 12 is a second partial schematic structural diagram of a portable system for cold therapy with optional heat and compression therapy in the manner of the heat exchanger being the first cooling plate.

FIG. 13 is a partial exploded schematic structural structure of a portable system for cold therapy with optional heat and compression therapy in the manner of the heat exchanger being the first cooling plate.

FIG. 14 is a partial schematic structural diagram of a portable system for cold therapy with optional heat and compression therapy in a manner of the heat exchanger being a spiral copper tube.

FIG. 15 is a partial schematic structural diagram of a portable system for cold therapy with optional heat and compression therapy in a manner of the heat exchanger being a second cooling plate manner.

FIG. 16 is a first structural block diagram of a portable device for cold therapy with optional heat and compression therapy according to an embodiment of the present disclosure.

FIG. 17 is a second structural block diagram of a portable device for cold therapy with optional heat and compression therapy according to an embodiment of the present disclosure.

FIG. 18 is a first schematic structural diagram of a portable device for cold therapy with optional heat and compression therapy according to an embodiment of the present disclosure.

FIG. 19 is a second schematic structural diagram of a portable device for cold therapy with optional heat and compression therapy according to an embodiment of the present disclosure.

FIG. 20 is a first schematic structural diagram of a portable system for cold therapy with optional compression therapy according to an embodiment of the present disclosure.

FIG. 21 is a second schematic structural diagram of a portable system for cold therapy with optional compression therapy according to an embodiment of the present disclosure.

FIG. 22 is an exploded schematic structural diagram of a portable system for cold therapy with optional compression therapy according to an embodiment of the present disclosure.

FIG. 23 is a first partial schematic structural diagram of a portable system for cold therapy with optional compression therapy according to an embodiment of the present disclosure.

FIG. 24 is a second partial schematic structural diagram of a portable system for cold therapy with optional compression therapy according to an embodiment of the present disclosure.

FIG. 25 is a third partial schematic structural diagram of a portable system for cold therapy with optional compression therapy according to an embodiment of the present disclosure.

FIG. 26 is a fourth partial schematic structural diagram of a portable system for cold therapy with optional compression therapy according to an embodiment of the present disclosure.

FIG. 27 is a fifth partial schematic structural diagram of a portable system for cold therapy with optional compression therapy according to an embodiment of the present disclosure.

FIG. 28 is a sixth partial schematic structural diagram of a portable system for cold therapy with optional compression therapy according to an embodiment of the present disclosure.

FIG. 29 is a first schematic structural diagram of a portable device for cold therapy with optional compression therapy according to an embodiment of the present disclosure.

FIG. 30 is a second schematic structural diagram of a portable device for cold therapy with optional compression therapy according to an embodiment of the present disclosure.

FIG. 31 is a first structural block diagram of a portable device for cold therapy with optional compression therapy according to an embodiment of the present disclosure.

FIG. 32 is a second structural block diagram of a portable device for cold therapy with optional compression therapy according to an embodiment of the present disclosure.

FIG. 33 is a temperature profile on multiple surface locations of the body wrap according to an embodiment of the present disclosure.

FIG. 34 is a schematic structural diagram of a portable system for cold therapy with optional heat and compression therapy according to an embodiment of the present disclosure.

FIG. 35 is an exploded schematic structural diagram of a portable system for cold therapy with optional heat and compression therapy according to an embodiment of the present disclosure.

FIG. 36 is a first partial schematic structural diagram of a portable system for cold therapy with optional heat and compression therapy according to an embodiment of the present disclosure.

FIG. 37 is a second partial schematic structural diagram of a portable system for cold therapy with optional heat and compression therapy according to an embodiment of the present disclosure.

FIG. 38 is a third partial schematic structural diagram of a portable system for cold therapy with optional heat and compression therapy according to an embodiment of the present disclosure.

FIG. 39 is a fourth partial schematic structural diagram of a portable system for cold therapy with optional heat and compression therapy according to an embodiment of the present disclosure.

FIG. 40 is a schematic structural diagram of a portable device for cold therapy with optional heat and compression therapy according to an embodiment of the present disclosure.

FIG. 41 is a structural block diagram of a portable device for cold therapy with optional heat and compression therapy according to an embodiment of the present disclosure.

FIG. 42 is a cooling temperature profile on multiple surface locations of the body wrap according to an embodiment of the present disclosure.

FIG. 43 is a heating temperature profile on multiple surface locations of the body wrap according to an embodiment of the present disclosure.

FIG. 44 is a schematic structural diagram of a cold therapy device according to an embodiment of the present disclosure.

FIG. 45 is a schematic structural diagram of a cold therapy device according to another embodiment of the present disclosure.

FIG. 46 is a first schematic structural diagram of a first connector and a third connector of an encryption connection system according to an embodiment of the present disclosure.

FIG. 47 is a second schematic structural diagram of a first connector and a third connector of an encryption connection system according to an embodiment of the present disclosure.

FIG. 48 is a first schematic structural diagram of a first connector and a third connector of an encryption connection system according to another embodiment of the present disclosure.

FIG. 49 is a second schematic structural diagram of a first connector and a third connector of an encryption connection system according to another embodiment of the present disclosure.

FIG. 50 is a schematic structural diagram of a second connector and a fourth connector of an encryption connection system according to an embodiment of the present disclosure.

FIG. 51 is a schematic structural diagram of a second connector and a fourth connector of an encryption connection system according to another embodiment of the present disclosure.

FIG. 52 is a schematic diagram of functional modules of an encryption connection system according to an embodiment of the present disclosure.

FIG. 53 is a schematic diagram of functional modules of an encryption connection system according to another embodiment of the present disclosure.

### DETAILED DESCRIPTION

In order to enable the skilled person in the art to better understand technical solutions in the present disclosure, the technical solutions in the embodiments of the present disclosure will be described clearly and completely in combination with the accompanying drawings in the embodiments of the present disclosure. It is apparent that the described embodiments are only some of the embodiments, but not all the embodiments of the present disclosure. Based on the embodiments in the present disclosure, all other embodiments obtained by persons having ordinary skills in the art without creative works are within the protection scope of the present disclosure.

It should be noted that when a component is referred to as “fixed” or “placed on” another component, it is able to be directly or indirectly on the other component. When a component is referred to as “connected” to another component, it is able to be directly or indirectly connected to the other component.

In addition, the terms “first” and “second” are used for descriptive purposes only and are not to be understood as indicating or implying relative importance or as implicitly indicating the quantity of technical features indicated. Thus, a feature defined as “first” or “second” may explicitly or implicitly include one or more such features. In the description of the present disclosure, “plurality of” or “several” means two or more unless otherwise expressly specified.

It should be noted that structures, proportions, sizes, etc., depicted in the accompanying drawings are provided only to facilitate the comprehension and reading of the disclosed contents for those skilled in the art. They are not intended to limit the conditions under which the present disclosure able to be implemented and do not hold technical significance. Any modifications to the structures, changes in proportions, or adjustments in size that do not affect the effectiveness and objectives achievable by the present disclosure should still fall within the scope of the technical contents disclosed in the present disclosure. We list numbering of the components in the figures as below.

41: Accessory module; 411: Third connector; 412: Authentication carrier; 4121: Second wireless unit; 4122: Power supply unit; 42: Host module; 421: Fourth connector; 422: First pin member; 423: Second pin member; 424: Main control board; 4241: Identification unit; 4242: Encryption algorithm unit; 4243: Comparison unit; 4244: Counting unit; 43: Processing module; 431: Encryption pipeline assembly; 4311: Encryption pipeline; 4312: First connector; 4313: Second connector; 4314: Power line; 4315: First signal line; 4316: Second signal line; 432: Circuit board; 4321: Information reading and writing unit; 4322: Signal antenna; 433: First wireless unit; 44: Cold therapy system; M40: Portables cold therapy device.

311: Reservoir; 3111: Liquid outlet of the reservoir; 321: First heat exchanger; 3211: Liquid inlet of the first heat exchanger; 3212: Liquid outlet of the first heat exchanger; 3213: Air inlet of the first heat exchanger; 3214: Air outlet of the first heat exchanger; 322: Compressor; 323: Condenser; 324: Fan; 331: Second heat exchanger; 3311: Liquid inlet of the second heat exchanger; 3312: Liquid outlet of the second heat exchanger; 332: Heating sheet; 341: First tube; 342: Second tube; 343: Third tube; 351: First multiple-way connector; 3511: First connector; 3512: Second connector; 3513: Third connector; 352: Multiple-way solenoid valve; 3521: Fourth connector; 3522: Fifth connector; 3523: Sixth connector; 353: Second multiple-way connector; 2531: Seventh connector; 3532: Eighth connector; 3533: Ninth connector; 3534: Tenth connector; 3535: Eleventh connector; 361: Water pump; 362: Air pump; 363: Air tube; 364: Two-way solenoid valve; 365: Pressure relief valve; 371: Liquid level detector; 372: Temperature sensor; 373: Water pressure sensor; 374: Air pressure sensor; 381: Main control board; 382: Display screen; 383: Power interface; 384: Battery; 391: Shell; 392: Handle; A30: Body wrap; A301: Liquid inlet of the body wrap; A302: Liquid outlet of the body wrap; M: Portables device for cold therapy with optional heat and compression therapy.

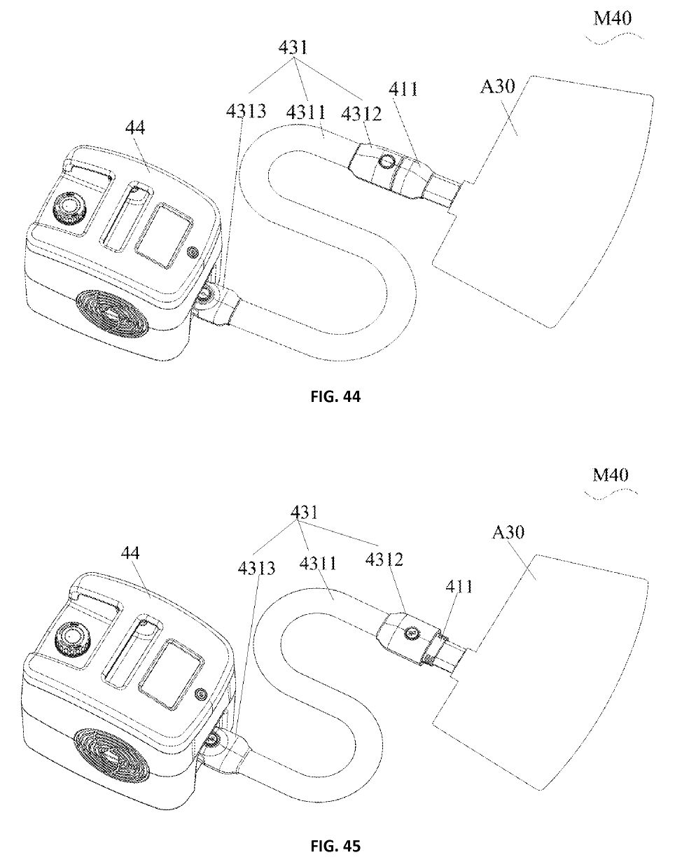

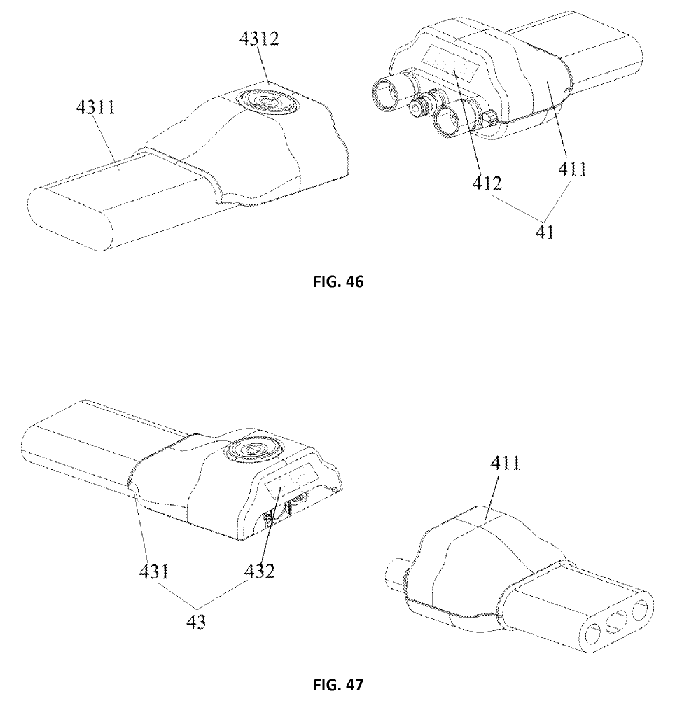

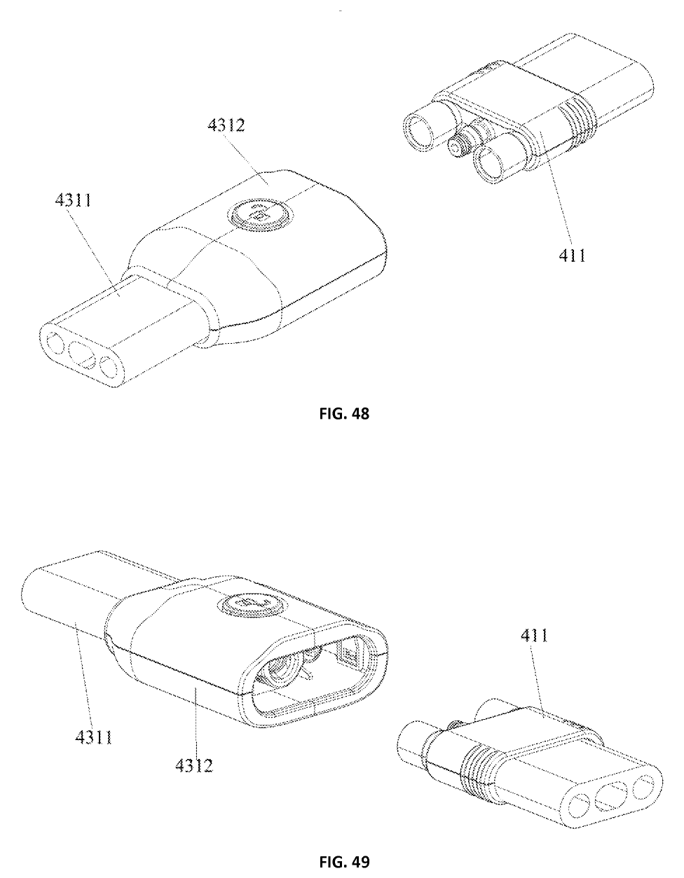

In a first aspect, referring to FIGS. 44-53, the present disclosure provides an encryption connection system, comprising: an accessory module 41, disposed on a body wrap A30 and configured to store identity information; a processing module 43, connected to the accessory module 41 and configured to obtain the identity information; and a host module 42, disposed in a cold therapy system 44 and connected to the processing module 43, configured to obtain the identity information through the processing module 43 and perform verification and matching; and wherein the host module 42 controls an operating state of the cold therapy system 44 according to a result of the verification and matching.

The encryption connection system according to the present disclosure has the following beneficial effects. The encryption connection system establishes a complete encrypted connection mechanism between the cold therapy system 44 and the body wrap A30. Through the coordinated operation of the accessory module 41, the processing module 43, and the host module 42, secure authentication based on identity information is achieved. Specifically, the accessory module 41 stores the identity information. The host module 42 obtains this identity information through the processing module 43 and performs matching verification. The cold therapy system 44 is only authorized to start treatment after the verification is passed. This design can accurately identify authorized body wraps A30, thereby effectively preventing the connection of unauthorized body wraps and ensuring the exclusivity and safety of the treatment. This encryption connection system fundamentally addresses issues such as medical operation risks, commercial infringement hazards, and data interference caused by the lack of a security authentication mechanism in traditional cold therapy devices, thereby enhancing the overall system's reliability and compliance.

In some embodiments, the accessory module comprises an authentication carrier; the identity information comprises an identification information, and the identification information is stored in the authentication carrier; and the host module comprises a main control board; the main control board comprises an identification unit, and the identification unit is configured to read the identification information to complete identification.

In some embodiments, the identity information further comprises a first authentication information, and the first authentication information is stored in the authentication carrier; and the main control board further comprises an encryption algorithm unit and a comparison unit; the encryption algorithm unit is configured to obtain the identification information and generate a second authentication information; the comparison unit is configured to compare the first authentication information and the second authentication information to complete a match.

In some embodiments, the main control board further comprises a counting unit; the counting unit is configured to record the usage count corresponding to each identification information.

In some embodiments, the processing module comprises a circuit board, and the circuit board is electrically connected to the main control board; and the circuit board comprises an information reading and writing unit and a signal antenna; wherein the information reading and writing unit is communicatively connected to the authentication carrier through the signal antenna; the information reading and writing unit is configured to write the first authentication information into the authentication carrier, and to read the identity information from the authentication carrier.

In some embodiments, the processing module further comprises an encryption pipeline assembly, configured to connect the cold therapy system and the body wrap; the encryption pipeline assembly comprises an encryption pipeline, a first connector, and a second connector, wherein the first connector and the second connector are respectively arranged at two ends of the encryption pipeline; and the accessory module further comprises a third connector; the host module further comprises a fourth connector; the encryption pipeline assembly is detachably connected to the body wrap via the first connector and the third connector; the encryption pipeline assembly is detachably connected to the cold therapy system via the second connector and the fourth connector.

In some embodiments, the information reading and writing unit and the signal antenna are both arranged inside or on the surface of the first connector; the authentication carrier is arranged inside or on the surface of the third connector; and the encryption pipeline assembly further comprises a power line and a first signal line, wherein both the power line and the first signal line are embedded in the pipe wall of the encryption pipeline; the main control board is electrically connected to the circuit board via the power line and the first signal line; and when the first connector is mated with the third connector, the signal antenna and the authentication carrier are spatially aligned to establish a near-field communication connection, enabling the information reading and writing unit to acquire the identity information stored in the authentication carrier.

In some embodiments, the power line and the first signal line pass through the second connector and protrude to form a first protrusion; the fourth connector is provided with a first pin member; the main control board is electrically connected to the first protrusion through the first pin member.

In some embodiments, the information reading and writing unit is arranged inside the cold therapy system; the signal antenna is arranged inside or on the surface of the first connector; the authentication carrier is arranged inside or on the surface of the third connector; and the encryption pipeline assembly further comprises a second signal line, wherein the second signal line is embedded in the pipe wall of the encryption pipeline; the information reading and writing unit is electrically connected to the signal antenna through the second signal line; when the first connector is mated with the third connector, the signal antenna and the authentication carrier are spatially aligned to establish a near-field communication connection, enabling the information reading and writing unit to acquire the identity information stored in the authentication carrier.

In some embodiments, the second signal line passes through the second connector and protrudes to form a second protrusion; the fourth connector is provided with a second pin member; the information reading and writing unit is electrically connected to the second protrusion through the second pin member.

In some embodiments, the information reading and writing unit and the signal antenna are both arranged inside the cold therapy system; the authentication carrier is arranged inside or on the surface of the third connector; when the third connector approaches the cold therapy system within a preset sensing distance range, the signal antenna and the authentication carrier establish a far-field communication connection through the spatial electromagnetic field, enabling the information reading and writing unit to acquire the identity information stored in the authentication carrier.

In some embodiments, the processing module comprises a first wireless unit, and the first wireless unit is electrically connected to the main control board; and the authentication carrier comprises a second wireless unit; the first wireless unit and the second wireless unit are connected via wireless communication; the first wireless unit is configured to write the first authentication information into the second wireless unit, and to read the identity information from the second wireless unit.

In some embodiments, the authentication carrier further comprises a power supply unit; the power supply unit is electrically connected to the second wireless unit and configured to supply operating power to the second wireless unit; and wherein the power supply unit is a rechargeable battery, a disposable battery, or a charging receiver board.

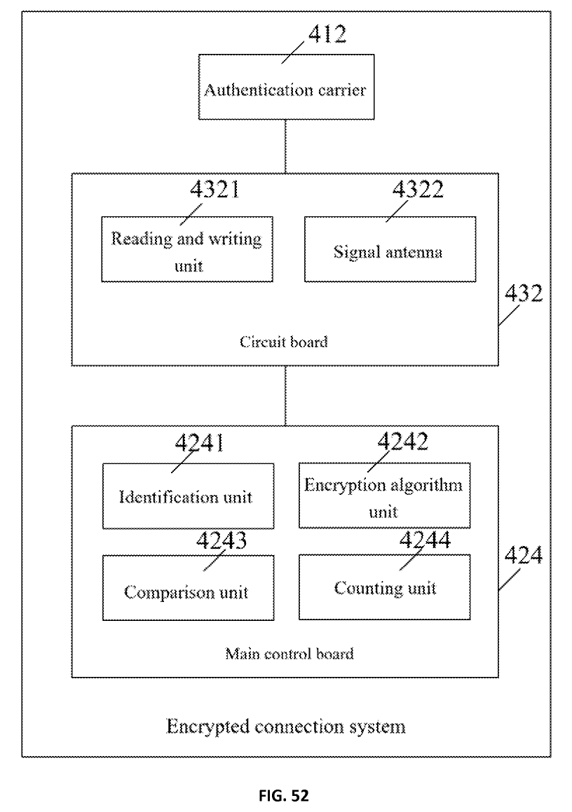

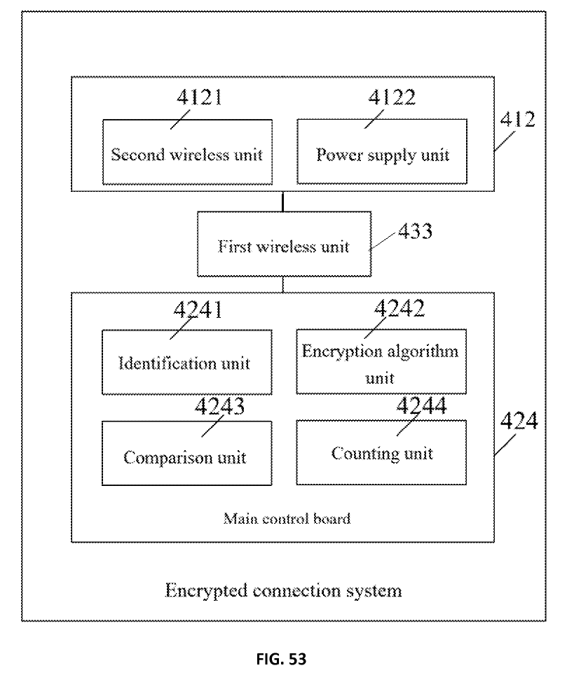

In one embodiment, referring to FIGS. 52-53, the accessory module 41 comprises an authentication carrier 412; the identity information comprises identification information, and the identification information is stored in the authentication carrier 412; and the host module 42 comprises a main control board 424; the main control board 424 comprises an identification unit 4241, and the identification unit 4241 is configured to read the identification information to complete identification.

Specifically, the identification information may be a unique, unalterable, and non-replicable ID number solidified within the authentication carrier 412. This unique ID number is written into the authentication carrier 412 during its production, forming a proprietary physical identifier for each body wrap A30, and serves as the ultimate basis for identity authentication and accessory traceability in the encryption connection.

More specifically, the method for the identification unit 4241 to verify the legality of the ID number may be selected from the following: first, comparing it against a pre-stored list of legal ID numbers on the main control board 424; second, verifying whether the number of digits in the ID number conforms to a preset 20-digit identifier.

In one embodiment, referring to FIGS. 52-53, the identity information further comprises first authentication information, and the first authentication information is stored in the authentication carrier 412; and the main control board 424 further comprises an encryption algorithm unit 4242 and a comparison unit 4243; the encryption algorithm unit 4242 is configured to obtain the identification information and generate second authentication information; the comparison unit 4243 is configured to compare the first authentication information and the second authentication information to complete a match.

Specifically, the first authentication information may be a first password generated by processing the ID number using a specific encryption algorithm and pre-installed in the authentication carrier 412. The encryption algorithm unit 4242 has built-in logic identical to that specific encryption algorithm. Its workflow is as follows: after the identification unit 4241 confirms the ID number is correct, the encryption algorithm unit 4242 generates a second password. The comparison unit 4243 then compares the first password with the second password. If they match, the body wrap A30 is judged as legal, and authorization is granted to establish an encrypted connection. If they do not match, the body wrap A30 is judged as illegal, and the connection is refused.

More specifically, the unique ID number is a 20-digit identifier, and the first password is a 6-digit numeric key obtained by performing pairwise XOR operations on four specific numerical values extracted from the ID number.

More specifically, the comparison unit 4243 may be a password comparison chip, used to logically compare the first password and the second password, generating either a correct or an incorrect verification result. Here, a correct verification result may correspond to a high-level signal, and an incorrect verification result may correspond to a low-level signal. The main control board 424 then determines whether to initiate treatment based on the signal.

In one embodiment, referring to FIGS. 52-53, the main control board 424 further comprises a counting unit 4244; the counting unit 4244 is configured to record the usage count corresponding to each identification information.

Specifically, the main control board 424 further comprises a counting unit 4244. It establishes an independent counter for each body wrap A30 with a unique ID number, recording its usage count in real-time. This enables digital, targeted control over the lifecycle of individual body wraps A30, effectively preventing usage beyond established limits.

In one embodiment, referring to FIG. 52, the processing module 43 comprises a circuit board 432, and the circuit board 432 is electrically connected to the main control board 424; and the circuit board 432 comprises an information reading and writing unit 4321 and a signal antenna 4322; wherein the information reading and writing unit 4321 is communicatively connected to the authentication carrier 412 through the signal antenna 4322; the information reading and writing unit 4321 is configured to write the first authentication information into the authentication carrier 412, and to read the identity information from the authentication carrier 412.

Specifically, the information reading and writing unit 4321 and the signal antenna 4322 serve as a bridge between the main control board 424 and the authentication carrier 412. Their workflow comprises two stages: initialization writing and runtime reading. During the initialization writing stage, the encryption algorithm unit 4242 generates the first password based on the ID number and commands the information reading and writing unit 4321 to securely write it into a designated storage area of the authentication carrier 412. During the runtime reading stage, the information reading and writing unit 4321 is responsible for driving the signal antenna 4322 to read the ID number and the first password stored in the authentication carrier 412, and transmitting them completely and accurately to the main control board 424 to complete the subsequent verification process.

In one embodiment, referring to FIGS. 44-51, the processing module 43 further comprises an encryption pipeline assembly 431, configured to connect the cold therapy system 44 and the body wrap A30; the encryption pipeline assembly 431 comprises an encryption pipeline 4311, a first connector 4312, and a second connector 4313, wherein the first connector 4312 and the second connector 4313 are respectively arranged at two ends of the encryption pipeline 4311; and the accessory module 41 further comprises a third connector 411; the host module 42 further comprises a fourth connector 421; the encryption pipeline assembly 431 is detachably connected to the body wrap A30 via the first connector 4312 and the third connector 411; the encryption pipeline assembly 431 is detachably connected to the cold therapy system 44 via the second connector 4313 and the fourth connector 421.

Specifically, the first connector 4312, the second connector 4313, the third connector 411, or the fourth connector 421 is integrated with a mechanical or magnetic locking mechanism. The mechanism automatically locks upon correct insertion to prevent accidental disconnection during use. A specific operating force must be applied for unlocking, achieving a balance between safety and convenience.

In one embodiment, referring to FIGS. 44-50 and 52, the information reading and writing unit 4321 and the signal antenna 4322 are both arranged inside or on the surface of the first connector 4312; the authentication carrier 412 is arranged inside or on the surface of the third connector 411; and the encryption pipeline assembly 431 further comprises a power line 4314 and a first signal line 4315, wherein both the power line 4314 and the first signal line 4315 are embedded in the pipe wall of the encryption pipeline 4311; the main control board 424 is electrically connected to the circuit board 432 via the power line 4314 and the first signal line 4315; and when the first connector 4312 is mated with the third connector 411, the signal antenna 4322 and the authentication carrier 412 are spatially aligned to establish a near-field communication connection, enabling the information reading and writing unit 4321 to acquire the identity information stored in the authentication carrier 412.

Specifically, this embodiment provides a highly integrated and reliable physical implementation scheme for near-field communication. The information reading and writing unit 4321 is specifically a near-field controller chip (such as an NFC controller chip), and the signal antenna 4322 is specifically a near-field communication antenna (such as an NFC coil antenna). The information reading and writing unit 4321 and the signal antenna 4322 are arranged in a modular form inside or on the surface of the first connector 4312. The authentication carrier 412 is specifically a near-field communication electronic tag (such as an NFC tag chip), which is arranged in a modular form inside or on the surface of the third connector 411. When the first connector 4312 and the third connector 411 are axially mated until mechanically locked in place, the signal antenna 4322 and the authentication carrier 412 are automatically precisely aligned in space, and the distance between them is confined within the effective working range for near-field communication, thereby creating the necessary physical conditions for stable data exchange.

More specifically, the main control board 424 supplies power to the circuit board 432 via the power line 4314 and transmits signals to the circuit board 432 via the first signal line 4315. Both the power line 4314 and the first signal line 4315 forming the electrical connection between them are embedded within the wall of the encryption pipeline 4311. This achieves encapsulation protection and electrical isolation for the physical layer of the connection link, effectively enhancing the reliability and anti-interference capability of data transmission while simplifying external wiring.

More specifically, the power line 4314 comprises two metal wires: a positive wire and a negative wire. One end of both wires is soldered to the power interface of the circuit board 432, and the other end is electrically connected to the main control board 424. This enables the main control board 424 to provide a stable operating voltage to the circuit board 432, ensuring the circuit board's password reading and storage functions.

More specifically, the first signal line 4315 comprises at least one metal wire. Even more specifically, it can be configured as two wires: a transmission wire and a reception wire. One end of the transmission wire is connected to the signal transmission terminal of the main control board 424, and the other end is connected to the signal reception terminal of the circuit board 432, used for transmitting signals from the main control board 424, such as password reading commands. One end of the reception wire is connected to the signal transmission terminal of the circuit board 432, and the other end is connected to the signal reception terminal of the main control board 424, used for transmitting feedback signals from the circuit board 432, such as the body wrap's A30 ID number and first password.

The workflow is briefly summarized as follows: When the user inserts the third connector 411 of the body wrap A30 into the first connector 4312 of the encryption pipeline assembly 431, the signal antenna 4322 and the authentication carrier 412 enter a communication-ready state simultaneously with the completion of the mechanical connection. After the host module 42 is powered on, the main control board 424 drives the signal antenna 4322 of the first connector 4312 via the first signal line 4315 and the information reading and writing unit 4321, transmitting radio frequency energy and read commands to the authentication carrier 412 of the third connector 411. Once activated, the authentication carrier 412 sends back the unique ID number and first password stored within it. This information returns to the main control board 424 via the original path, initiating the subsequent identification, encryption operation, and comparison authentication processes.

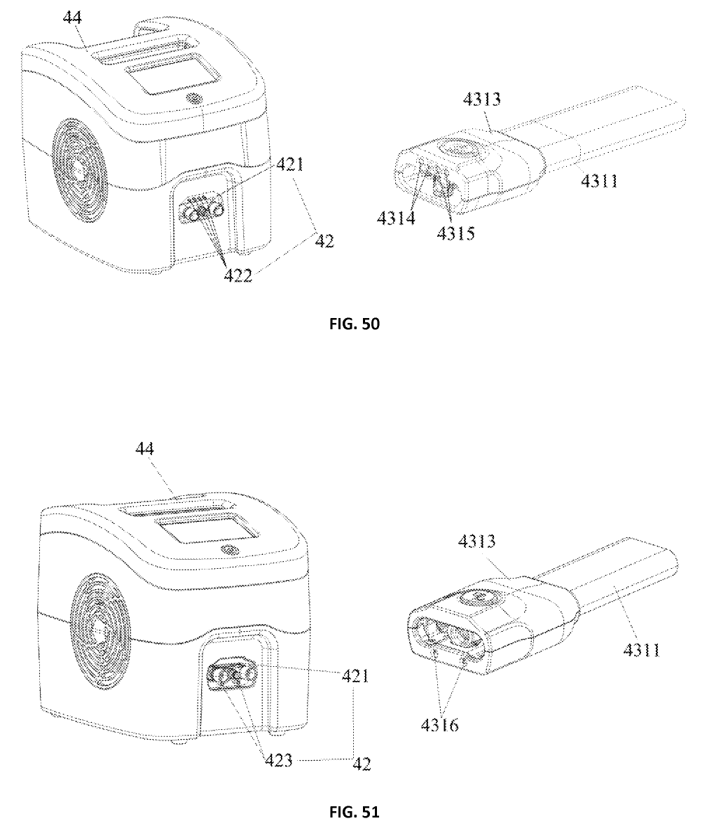

In one embodiment, referring to FIG. 50, the power line 4314 and the first signal line 4315 pass through the second connector 4313 and protrude to form a first protrusion; the fourth connector 421 is provided with a first pin member 422; the main control board 424 is electrically connected to the first protrusion through the first pin member 422.

Specifically, the first pin member 422 is a metal conductive pin, which can be a gold-plated copper pin, fixedly embedded inside the fourth connector 421. The number of these pins corresponds to the total number of power lines 4314 and first signal lines 4315. When the second connector 4313 is mated with the fourth connector 421, one end of the first pin member 422 makes tight contact with the first protrusion, while the other end is electrically connected to the main control board 424 via wires inside the cold therapy system 44. This ultimately establishes the power supply circuit and data communication circuit among the main control board 424, the first pin member 422, the first protrusion, the power line 4314, the first signal line 4315, the information reading and writing unit 4321, and the signal antenna 4322.

In one embodiment, referring to FIGS. 44-49, 51 and 52, the information reading and writing unit 4321 is arranged inside the cold therapy system 44; the signal antenna 4322 is arranged inside or on the surface of the first connector 4312; the authentication carrier 412 is arranged inside or on the surface of the third connector 411; and the encryption pipeline assembly 431 further comprises a second signal line 4316, wherein the second signal line 4316 is embedded in the pipe wall of the encryption pipeline 4311; the information reading and writing unit 4321 is electrically connected to the signal antenna 4322 through the second signal line 4316; when the first connector 4312 is mated with the third connector 411, the signal antenna 4322 and the authentication carrier 412 are spatially aligned to establish a near-field communication connection, enabling the information reading and writing unit 4321 to acquire the identity information stored in the authentication carrier 412.

Specifically, this embodiment provides a highly integrated and reliable physical implementation scheme for near-field communication. The information reading and writing unit 4321 is specifically a near-field controller chip (such as an NFC controller chip) and is preferably disposed on the main control board 424. The signal antenna 4322 is specifically a near-field communication antenna (such as an NFC coil antenna) and is arranged in a modular form inside or on the surface of the first connector 4312. The authentication carrier 412 is specifically a near-field communication electronic tag (such as an NFC tag chip) and is arranged in a modular form inside or on the surface of the third connector 411. When the first connector 4312 and the third connector 411 are axially mated until mechanically locked in place, the signal antenna 4322 and the authentication carrier 412 are automatically precisely aligned in space, and the distance between them is confined within the effective working range for near-field communication, thereby creating the necessary physical conditions for stable data exchange.

More specifically, the second signal line 4316 comprises at least one metal wire. Even more specifically, it can be configured as two wires: a transmission wire and a reception wire. One end of the transmission wire is connected to the signal transmission terminal of the information reading and writing unit 4321, and the other end is connected to the signal reception terminal of the signal antenna 4322, used for transmitting signals from the main control board 424, such as password reading commands, through the information reading and writing unit 4321 to the signal antenna 4322. One end of the reception wire is connected to the signal transmission terminal of the signal antenna 4322, and the other end is connected to the signal reception terminal of the information reading and writing unit 4321, used for transmitting feedback signals from the signal antenna 4322 through the information reading and writing unit 4321 to the main control board 424. By arranging the information reading and writing unit 4321 inside the cold therapy system 44, only the second signal line 4316 needs to be provided within the encryption pipeline 4311, eliminating the need for a power line. This simplifies the internal wiring while further enhancing the reliability of data transmission.

The workflow is briefly summarized as follows: When the user inserts the third connector 411 of the body wrap A30 into the first connector 4312 of the encryption pipeline assembly 431, the signal antenna 4322 and the authentication carrier 412 enter a communication-ready state simultaneously with the completion of the mechanical connection. After the host module 42 is powered on, the main control board 424 drives the signal antenna 4322 of the first connector 4312 via the information reading and writing unit 4321 and the second signal line 4316, transmitting radio frequency energy and read commands to the authentication carrier 412 of the third connector 411. Once activated, the authentication carrier 412 sends back the unique ID number and first password stored within it. This information returns to the main control board 424 via the original path, initiating the subsequent identification, encryption operation, and comparison authentication processes.

In one embodiment, referring to FIG. 51, the second signal line 4316 passes through the second connector 4313 and protrude to form a second protrusion; the fourth connector 421 is provided with a second pin member 423; the information reading and writing unit 4321 is electrically connected to the second protrusion through the second pin member 423.

Specifically, the second pin member 423 is a metal conductive pin, which can be a gold-plated copper pin, fixedly embedded inside the fourth connector 421. The number of these pins corresponds to the total number of second signal lines 4316. When the second connector 4313 is mated with the fourth connector 421, one end of the second pin member 423 makes tight contact with the second protrusion, while the other end is electrically connected to the information reading and writing unit 4321 via wires inside the cold therapy system 44. This ultimately establishes the power supply circuit and data communication circuit among the main control board 424, the information reading and writing unit 4321, the second pin member 423, the second protrusion, the second signal line 4316, and the signal antenna 4322.

In one embodiment, referring to FIGS. 44, 45 and 52, the information reading and writing unit 4321 and the signal antenna 4322 are both arranged inside the cold therapy system 44; the authentication carrier 412 is arranged inside or on the surface of the third connector 411; when the third connector 411 approaches the cold therapy system 44 within a preset sensing distance range, the signal antenna 4322 and the authentication carrier 412 establish a far-field communication connection through the spatial electromagnetic field, enabling the information reading and writing unit 4321 to acquire the identity information stored in the authentication carrier 412.

Specifically, this embodiment provides a non-contact, flexible recognition-distance far-field communication physical implementation scheme. The information reading and writing unit 4321 is specifically a far-field controller chip (such as a UHF RFID control chip) and is preferably disposed on the main control board 424. The signal antenna 4322 is specifically a far-field communication antenna (such as a UHF RFID antenna) and is arranged as an independent component on the inner sidewall of the cold therapy system 44. The authentication carrier 412 is specifically a far-field communication electronic tag (such as a UHF RFID tag) and is arranged in a modular form inside or on the surface of the third connector 411. When the third connector 411 is within the preset sensing distance range of the cold therapy system 44, the far-field communication antenna automatically radiates a far-field electromagnetic wave signal, which wirelessly pairs and exchanges information with the far-field communication electronic tag built into the authentication carrier 412 via the spatial electromagnetic field. This enables reliable reading of identity information without the need for physical alignment, and eliminates the need for wires within the encryption pipeline, simplifying internal wiring while further enhancing the reliability of data transmission.

The workflow is briefly summarized as follows: When the user brings the third connector 411 within the preset sensing distance of the cold therapy system 44, a far-field communication link is automatically established between the far-field communication antenna and the authentication carrier 412. The main control board 424 drives the far-field communication antenna, via the information reading and writing unit 4321, to continuously transmit far-field electromagnetic waves and broadcast read commands. The authentication carrier 412 within the effective range is activated by the far-field electromagnetic waves, modulates the unique ID number and first password stored within it, and backscatters them to the far-field communication antenna. This modulated signal is decoded by the information reading and writing unit 4321 and then transmitted to the main control board 424, initiating the subsequent identification, decryption operation, and comparison authentication processes.

In one embodiment, referring to FIGS. 44, 45, and 53, the processing module 43 comprises a first wireless unit 433, and the first wireless unit 433 is electrically connected to the main control board 424; and the authentication carrier 412 comprises a second wireless unit 4121; the first wireless unit 433 and the second wireless unit 4121 are connected via wireless communication; the first wireless unit 433 is configured to write the first authentication information into the second wireless unit 4121, and to read the identity information from the second wireless unit 4121.

Specifically, this embodiment provides a two-way authentication implementation scheme based on a general-purpose wireless communication protocol. The first wireless unit 433 may be a Bluetooth, Wi-Fi, ZigBee, or cellular mobile communication unit that complies with a preset communication standard, integrated on the main control board 424 or electrically connected to the main control board 424 as an independent module. The second wireless unit 4121 is a wireless communication chip or module that matches the communication protocol of the first wireless unit 433, embedded inside the third connector 411. The first wireless unit 433 and the second wireless unit 4121 establish a long-term or temporary encrypted wireless connection after initial pairing, supporting two-way data exchange. In addition to reading identity information such as the pre-stored unique ID number from the second wireless unit 4121, the first wireless unit 433 can also write the first password generated by the host module 42 into it.

The workflow is briefly summarized as follows: When the third connector 411 is first connected to the system, the user needs to trigger a pairing operation, causing the main control board 424 to control the first wireless unit 433 to enter discovery mode, search for and complete the initial pairing with the second wireless unit 4121, and write the generated first password into the second wireless unit 4121. Thereafter, during regular use, when the third connector 411 is within the communication range of the first wireless unit 433, the two automatically establish an encrypted connection. The first wireless unit 433 reads the identity information stored in the second wireless unit 4121 and transmits it to the main control board 424, initiating the subsequent identification, decryption operation, and comparison authentication processes.

In one embodiment, referring to FIG. 53, the authentication carrier 412 further comprises a power supply unit 4122; the power supply unit 4122 is electrically connected to the second wireless unit 4121 and configured to supply operating power to the second wireless unit 4121; and wherein the power supply unit 4122 is a rechargeable battery, a disposable battery, or a charging receiver board.

Specifically, when the wireless communication connection employs bidirectional radio frequency communication methods such as Bluetooth, Wi-Fi, or cellular mobile communication, the second wireless unit 4121 is an active radio frequency module requiring continuous power supply. The power supply unit 4122 provides stable operating power to the second wireless unit 4121, enabling it to autonomously complete signal reception, processing, modulation, and transmission, and to support complex bidirectional authentication protocols and data exchange processes. This ensures the reliability and real-time responsiveness of the communication link while achieving contactless identification.

In a second aspect, referring to FIGS. 44-53, the present disclosure provides an encryption connection method, comprising: initiating an identity authentication request from a host module 42 of a cold therapy system 44 to a processing module 43; the processing module 43, according to the identity authentication request, acquiring identity information stored in an accessory module 41 of a body wrap A30; the host module 42 receiving the identity information returned by the processing module 43, and performing verification and matching on the identity information based on a preset verification rule; the host module 42 generating a control instruction according to a verification and matching result to control an operating state of the cold therapy system 44; wherein, when the verification and matching is successful, the host module 42 authorizes the cold therapy system 44 to start operation; when the verification and matching fails, the host module 42 maintains the cold therapy system 44 in a locked or off state.

The encryption connection method according to the present disclosure has the following beneficial effects. The encryption connection method provides a hierarchical, clear, secure, and reliable authentication control process. This method involves the host module 42 actively initiating an authentication request, driving the processing module 43 to acquire identity information from the accessory module 41, and then having the host module 42 complete verification and matching based on preset rules. Finally, the operating state of the cold therapy system 44 is determined according to the matching result. This process transforms the hardware authentication mechanism into standardized software logic steps, ensuring that only authorized body wraps A30 that pass strict verification can trigger the treatment procedure, thereby achieving security control over the medical process at the operational level. The method is rigorous in process, clear in judgment, and easy to integrate into existing cold therapy device control systems. It can effectively prevent unauthorized use and ensure the compliance and controllability of treatment.

In some embodiments, the identity information comprises an identification information and a first authentication information; and the step of verification and matching comprises: the identification unit of the host module reading the identification information stored in the authentication carrier of the accessory module to complete identification; the encryption algorithm unit of the host module generating a second authentication information based on the identification information; the comparison unit of the host module comparing the first authentication information and the second authentication information to complete authentication based on the comparison result.

In some embodiments, the communication method between the processing module and the accessory module comprises wireless communication connection or wired electrical connection; and wherein the wireless communication connection comprises short-range wireless communication based on inductive coupling, including NFC or HF RFID; or long-range wireless communication based on electromagnetic wave radiation, including UHF RFID, Bluetooth, Wi-Fi, ZigBee, or cellular mobile communication.

In one embodiments, referring to FIGS. 52-53, the identity information comprises identification information and first authentication information; and the step of verification and matching comprises: the identification unit 4241 of the host module 42 reading the identification information stored in the authentication carrier 412 of the accessory module 41 to complete identification; the encryption algorithm unit 4242 of the host module 42 generating second authentication information based on the identification information; the comparison unit 4243 of the host module 42 comparing the first authentication information and the second authentication information to complete authentication based on the comparison result.

Specifically, the specific implementation steps of the encryption verification are further detailed. By clearly adopting the three sequential technical steps of identification, encrypted generation, and comparison, the identity authentication process is transformed into a rigorous chain of computation and validation from identification information to dynamic authentication information. This effectively prevents the risk of identity information being copied or forged, thereby further enhancing the security level of the entire cold therapy system 44.

In one embodiments, referring to FIGS. 52-53, the communication method between the processing module 43 and the accessory module 41 comprises wireless communication connection or wired electrical connection; and wherein the wireless communication connection comprises short-range wireless communication based on inductive coupling, including NFC or HF RFID; or long-range wireless communication based on electromagnetic wave radiation, including UHF RFID, Bluetooth, Wi-Fi, ZigBee, or cellular mobile communication.

Specifically, by explicitly covering multiple wireless and wired communication methods, flexible, reliable, and scalable technical implementation paths are provided for the connection between the processing module 43 and the accessory module 41. Short-range wireless communication based on inductive coupling (such as NFC/HF RFID) can provide highly secure and precisely triggered identity authentication. Long-range wireless communication based on electromagnetic wave radiation (such as UHF RFID, Bluetooth, Wi-Fi, ZigBee, or cellular mobile communication) supports convenient contactless, long-distance identification and data transmission. Wired electrical connections ensure absolute stability of communication in complex electromagnetic environments. This multi-modal communication support mechanism enables the system to adapt to requirements of different security levels, usage scenarios, and environmental conditions, significantly enhancing the product's practicality and compatibility.

In a third aspect, referring to FIGS. 34-53, the present disclosure provides a portable cold therapy device M40, comprising: a cold therapy system 44; a body wrap A30; and an encryption connection system, the encryption connection system comprising: an accessory module 41, disposed on the body wrap A30 and configured to store identity information; a processing module 43, connected to the accessory module 41 and configured to acquire the identity information; and a host module 42, disposed in the cold therapy system 44 and connected to the processing module 43, configured to acquire the identity information through the processing module 43 and perform verification and matching; and wherein the host module 42 controls an operating state of the cold therapy system 44 according to a result of the verification and matching.

The portable cold therapy device M40 according to the present disclosure has the following beneficial effects. The portable cold therapy device M40 integrates the encryption connection system as a core security component deeply between the cold therapy system 44 and the body wrap A30 of the portable cold therapy device M40, achieving device-level integrated security protection. Through the coordination of the built-in accessory module 41, processing module 43, and host module 42, the device completes a full hardware-level closed loop from identity identification and verification to operation control, ensuring that the device only functions normally when paired with an authorized and legitimate body wrap A30. This deeply integrated design fundamentally eliminates the possibility of using unauthorized body wraps, enhancing the product's own safety and reliability while also strengthening the exclusivity of its business model.

In some embodiments, the cold therapy system comprises a cooling mechanism and a reservoir; the cooling mechanism comprises a cooling component and a first heat exchanger, and the cooling component is used for cooling the first heat exchanger; the host module is electrically connected to the cooling component; the cooling mechanism is placed outside the reservoir; and wherein: the reservoir comprises a liquid outlet of the reservoir; the first heat exchanger comprises a liquid inlet of the first heat exchanger, and a liquid outlet of the first heat exchanger; the liquid in the reservoir can flow into the first heat exchanger through the liquid outlet of the reservoir and the liquid inlet of the first heat exchanger; when the liquid in the reservoir flows into the first heat exchanger first, and then flows into the body wrap through the liquid outlet of the first heat exchanger and the liquid inlet of the body wrap; the liquid in the body wrap can flow into the first heat exchanger first through the liquid outlet of the body wrap and the liquid inlet of the first heat exchanger, and then flow into the body wrap again, or the liquid in the body wrap can flow back to the reservoir through the liquid outlet of the body wrap and the liquid outlet of the reservoir.

In some embodiments, the cold therapy system further comprises a heating mechanism; the heating mechanism comprises a heating component and a second heat exchanger, and the heating component is used for heating the second heat exchanger; the host module is electrically connected to the heating component; the heating mechanism is placed outside the reservoir; and wherein: the second heat exchanger comprises a liquid inlet of the second heat exchanger, and a liquid outlet of the second heat exchanger; the liquid in the reservoir can flow into the second heat exchanger through the liquid outlet of the reservoir and the liquid inlet of the second heat exchanger; when the liquid in the reservoir flows into the second heat exchanger first, and then flows into the body wrap through the liquid outlet of the second heat exchanger and the liquid inlet of the body wrap; the liquid in the body wrap can flow into the second heat exchanger first through the liquid outlet of the body wrap and the liquid inlet of the second heat exchanger, and then flow into the body wrap again, or the liquid in the body wrap can flow back to the reservoir through the liquid outlet of the body wrap and the liquid outlet of the reservoir.

In some embodiments, the processing module comprises an encryption pipeline assembly, the encryption pipeline assembly comprises an encryption pipeline, and the cold therapy system is connected to the body wrap through the encryption pipeline to achieve fluid circulation; wherein the encryption pipeline comprises an air tube; the cold therapy system further comprises an air pump and a two-way solenoid valve; wherein, the air pump is connected to the body wrap through the air tube, and the two-way solenoid valve is arranged on the air tube; the air pump and the two-way solenoid valve are both electrically connected to the host module; and the body wrap is configured to be inflated by the air pump, and the body wrap is configured to be deflated by the two-way solenoid valve.

In one embodiments, referring to FIGS. 34-43, the cold therapy system 44 comprises a cooling mechanism and a reservoir 311; the cooling mechanism comprises a cooling component 322 and a first heat exchanger 321, and the cooling component 322 is used for cooling the first heat exchanger 321; the host module 42 is electrically connected to the cooling component 322; the cooling mechanism is placed outside the reservoir 311; and wherein: the reservoir 311 comprises a liquid outlet of the reservoir 3111; the first heat exchanger 321 comprises a liquid inlet of the first heat exchanger 3211, and a liquid outlet of the first heat exchanger 3212; the liquid in the reservoir 311 can flow into the first heat exchanger 321 through the liquid outlet of the reservoir 3111 and the liquid inlet of the first heat exchanger 3211; when the liquid in the reservoir 311 flows into the first heat exchanger 321 first, and then flows into the body wrap A30 through the liquid outlet of the first heat exchanger 3212 and the liquid inlet of the body wrap A301; the liquid in the body wrap A30 can flow into the first heat exchanger 321 first through the liquid outlet of the body wrap A302 and the liquid inlet of the first heat exchanger 3211, and then flow into the body wrap A30 again, or the liquid in the body wrap A30 can flow back to the reservoir 311 through the liquid outlet of the body wrap A302 and the liquid outlet of the reservoir 3111.

Specifically, the host module 42 can control the activation or deactivation of the cooling component 322. The chilled liquid within the cold therapy system 44 can be transmitted into the body wrap A30 via the encryption pipeline 4311, thereby achieving liquid conduction between the cold therapy system 44 and the body wrap A30.

In one embodiments, referring to FIGS. 34-43, the cold therapy system 44 further comprises a heating mechanism; the heating mechanism comprises a heating component 332 and a second heat exchanger 331, and the heating component 332 is used for heating the second heat exchanger 331; the host module 42 is electrically connected to the heating component 332; the heating mechanism is placed outside the reservoir 311; and wherein: the second heat exchanger 331 comprises a liquid inlet of the second heat exchanger 3311, and a liquid outlet of the second heat exchanger 3312; the liquid in the reservoir 311 can flow into the second heat exchanger 331 through the liquid outlet of the reservoir 3111 and the liquid inlet of the second heat exchanger 3311; when the liquid in the reservoir 311 flows into the second heat exchanger 331 first, and then flows into the body wrap A30 through the liquid outlet of the second heat exchanger 3312 and the liquid inlet of the body wrap A301; the liquid in the body wrap A30 can flow into the second heat exchanger 331 first through the liquid outlet of the body wrap A302 and the liquid inlet of the second heat exchanger 3311, and then flow into the body wrap A30 again, or the liquid in the body wrap A30 can flow back to the reservoir 311 through the liquid outlet of the body wrap A302 and the liquid outlet of the reservoir 3111.

Specifically, the host module 42 can control the activation or deactivation of the heating component 332. The heated liquid within the cold therapy system 44 can be transmitted into the body wrap A30 via the encryption pipeline 4311, thereby achieving liquid conduction between the cold therapy system 44 and the body wrap A30.

In one embodiments, referring to FIGS. 34-43, the processing module 43 comprises an encryption pipeline assembly 431, the encryption pipeline assembly 431 comprises an encryption pipeline 4311, and the cold therapy system 44 is connected to the body wrap A30 through the encryption pipeline 4311 to achieve fluid circulation; wherein the encryption pipeline 4311 comprises an air tube; the cold therapy system 44 further comprises an air pump 362 and a two-way solenoid valve 364; wherein, the air pump 362 is connected to the body wrap A30 through the air tube, and the two-way solenoid valve 364 is arranged on the air tube; the air pump 362 and the two-way solenoid valve 364 are both electrically connected to the host module 42; and the body wrap A30 is configured to be inflated by the air pump 362, and the body wrap A30 is configured to be deflated by the two-way solenoid valve 364.

Specifically, the host module 42 can control the activation or deactivation of the air pump 362 and the two-way solenoid valve 364, thereby achieving inflation and deflation of the body wrap A30 between the cold therapy system 44 and the body wrap A30.

In some examples, referring to FIGS. 34 to 43, the present disclosure provides a portable system for cold therapy with optional heat and compression therapy, applied to a body wrap A30, comprising a cooling mechanism, a heating mechanism, a reservoir, and a control assembly; the cooling mechanism comprises a cooling component and a first heat exchanger 321, and the cooling component is used for cooling the first heat exchanger 321; the heating mechanism comprises a heating component and a second heat exchanger 331, and the heating component is used for heating the second heat exchanger 331; the control assembly is electrically connected to the cooling component and the heating component, respectively; the cooling component and the heating component are placed outside the reservoir 311, respectively; and wherein: the reservoir 311 comprises a liquid outlet of the reservoir 3111; the first heat exchanger 321 comprises a liquid inlet of the first heat exchanger 3211, and a liquid outlet of the first heat exchanger 3212; the second heat exchanger 331 comprises a liquid inlet of the second heat exchanger 3311, and a liquid outlet of the second heat exchanger 3312; the liquid in the reservoir 311 can flow into the first heat exchanger 321 through the liquid outlet of the reservoir 3111 and the liquid inlet of the first heat exchanger 3211, or flow into the second heat exchanger 331 through the liquid outlet of the reservoir 3111 and the liquid inlet of the second heat exchanger 3311.

When the liquid in the reservoir 311 flows into the first heat exchanger 321 first, and then flows into the body wrap A30 through the liquid outlet of the first heat exchanger 3212 and the liquid inlet of the body wrap A301; the liquid in the body wrap A30 can flow into the first heat exchanger 321 first through the liquid outlet of the body wrap A302 and the liquid inlet of the first heat exchanger 3211, and then flow into the body wrap A30 again, or the liquid in the body wrap A30 can flow back to the reservoir 311 through the liquid outlet of the body wrap A302 and the liquid outlet of the reservoir 3111.

When the liquid in the reservoir 311 flows into the second heat exchanger 331 first, and then flows into the body wrap A30 through the liquid outlet of the second heat exchanger 3312 and the liquid inlet of the body wrap A301; the liquid in the body wrap A30 can flow into the second heat exchanger 331 first through the liquid outlet of the body wrap A302 and the liquid inlet of the second heat exchanger 3311, and then flow into the body wrap A30 again, or the liquid in the body wrap A30 can flow back to the reservoir 311 through the liquid outlet of the body wrap A302 and the liquid outlet of the reservoir 3111.

It should be noted that the liquid includes water, alcohol, antifreezing solution, and the like. A concentration of 10% alcohol is preferably used.

It should be noted that the first heat exchanger and the second heat exchanger are brazed plate heat exchangers.

The technical problems to be solved in the embodiments lie in insufficient cooling effect, low efficiency and single function of the cooling mechanism.

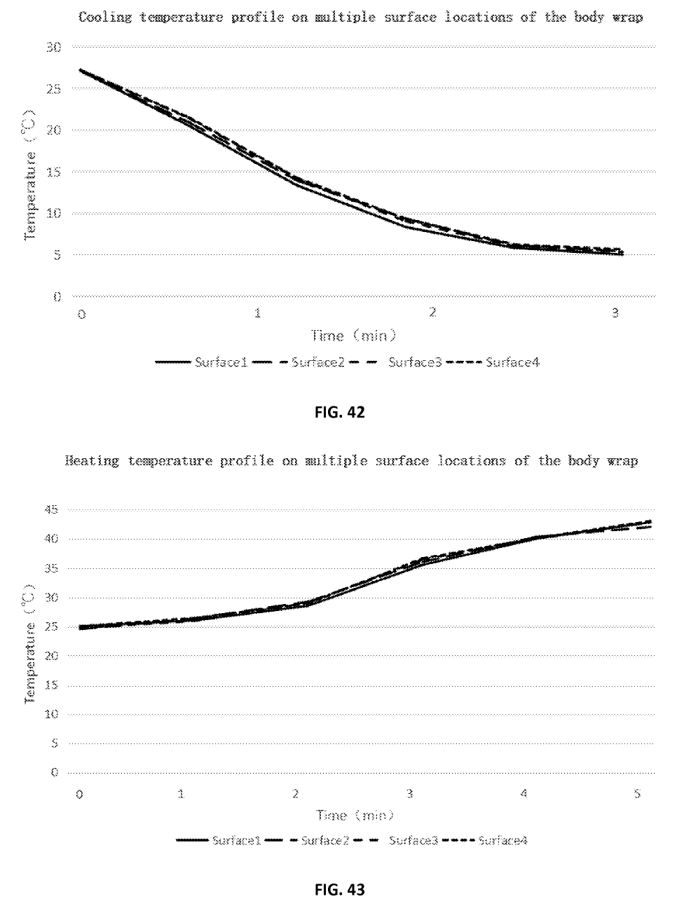

The portable system for cold therapy with optional heat and compression therapy according to the present disclosure has the following beneficial effects. In some examples, by setting the cooling component and the first heat exchanger, the first heat exchanger 321 can be cooled through the cooling component, the liquid in the reservoir 311 can flow into the body wrap A30 after cooling through the first heat exchanger 321, the liquid in the body wrap A30 can flow into the first heat exchanger 321 again for cooling, or the liquid in the body wrap A30 can flow back into the reservoir 311, providing the body wrap A30 with a cold compression function, and the remaining liquid in the reservoir 311 will no longer participate in the circulation, to greatly improve the cooling efficiency, and reduce the power consumption. Under the condition of the ambient temperature of 25-28℃, referring to FIG. 42, with an area of 1m² of the body wrap A30, a cooling rate is such that the surface temperature of the body wrap A30 reaches 5℃ within about 3 minutes, a cooling power is about 75 watts, and the achieved cooling capacity is about 250 watts. The device exhibits excellent cooling effects, high efficiency, and stability. In some examples, by setting the heating component and the second heat exchanger 331, the second heat exchanger 331 can be heated through the heating component, the liquid in the reservoir 311 can flow into the body wrap A30 after heating through the second heat exchanger 331, the liquid in the body wrap A30 can flow into the second heat exchanger 331 again for heating, or the liquid in the body wrap A30 can flow back into the reservoir 311, providing the body wrap A30 with a hot compression function, and the remaining liquid in the reservoir 311 will no longer participate in the circulation, to greatly improve the heating efficiency, and reduce the power consumption. Under the condition of the ambient temperature of 25-28℃, referring to FIG. 43, with an area of 1m² of the body wrap A30, a heating rate is such that the surface temperature of the body wrap A30 reaches 43℃ within about 5 minutes, a heating power is about 150 watts, and the achieved heating capacity is about 150 watts. The device exhibits excellent heating effects, high efficiency, and stability. The cooling and heating mechanisms are simultaneously incorporated into the portable device for cold therapy with optional heat and compression therapy, provided in the present disclosure. The device has a compact, small, and lightweight structure, facilitating convenient portability.

Furthermore, the heat exchanger of the cooling mechanism and the heating mechanism are set separately, so that the first heat exchanger 321 allows pre-cooling, and the second heat exchanger 331 allows pre-heating, thereby enabling immediate use when the user requires therapy.

In some embodiments, the cooling component comprises a compressor, a condenser, and a fan, and the control assembly is electrically connected to the compressor and the fan, respectively; and wherein: the first heat exchanger comprises an air inlet of the first heat exchanger, an air outlet of the first heat exchanger; the compressor has an air inlet connected to the air outlet of the first heat exchanger through a first tube; the air inlet of the first heat exchanger is connected to the condenser through a second tube; the condenser is connected to an air outlet of the compressor through a third tube; the first tube and the second tube are connected by fluid, and the second tube and the third tube are connected by fluid, enabling refrigerant in the compressor to pass through the condenser first and then through the first heat exchanger to achieve a cooling effect; the fan has a working surface facing the condenser.

In some embodiments, the heating component comprises one or more heating sheets, which are arranged on the outside of the second heat exchanger; the one or more heating sheets are electrically connected to the control assembly.

In some embodiments, further comprising a insulation device, the insulation device is arranged on the outside of the heating sheet.

In some embodiments, further comprising a first multiple-way connector, wherein the first multiple-way connector comprises a first connector, a second connector, and a third connector; wherein the first connector is connected to the liquid outlet of the reservoir, the second connector is connected to the liquid inlet of the first heat exchanger and the liquid inlet of the second heat exchanger, the third connector is connected to the liquid outlet of the body wrap.

In some embodiments, further comprising a multiple-way solenoid valve, which is electrically connected to the control assembly; wherein the multiple-way solenoid valve comprises a fourth connector, a fifth connector, and a sixth connector; wherein the fourth connector is connected to the second connector, the fifth connector is connected to the liquid inlet of the first heat exchanger, the sixth connector is connected to the liquid inlet of the second heat exchanger.

In some embodiments, further comprising a second multiple-way connector, wherein the second multiple-way connector comprises a seventh connector, a eighth connector, and a ninth connector; wherein the seventh connector is connected to the liquid outlet of the first heat exchanger, the eighth connector is connected to the liquid outlet of the second heat exchanger, the ninth connector is connected to the liquid inlet of the body wrap.

In some embodiments, further comprising a water pump is arranged on a tube connected between the second connector and the fourth connector, and the water pump is electrically connected to the control assembly; and wherein, when the fourth connector and the fifth connector are connected, in response to the water pump working, the liquid in the reservoir flows into the first heat exchanger through the first multiple-way connector and the multiple-way solenoid valve, and then flows into the body wrap through the second multiple-way connector; the liquid in the body wrap flows into the first heat exchanger through the first multiple-way connector and the multiple-way solenoid valve, and then flows into the body wrap again through the second multiple-way connector; when the fourth connector and the sixth connector are connected, in response to the water pump working, the liquid in the reservoir flows into the second heat exchanger through the first multiple-way connector and the multiple-way solenoid valve, and then flows into the body wrap through the second multiple-way connector; the liquid in the body wrap flows into the second heat exchanger through the first multiple-way connector and the multiple-way solenoid valve, and then flows into the body wrap again through the second multiple-way connector; in response to the water pump stopped working, the liquid in the body wrap flows back to the reservoir through the first multiple-way connector.

In some embodiments, the reservoir is further provided with a liquid level detector, and the liquid level detector is electrically connected to the control assembly.

In some embodiments, the second multiple-way connector further comprises a tenth connector, the tenth connector is connected with a temperature sensor, and the temperature sensor is electrically connected to the control assembly.

In some embodiments, the second multiple-way connector further comprises a eleventh connector, the eleventh connector is connected with a water pressure sensor, and the water pressure sensor is electrically connected to the control assembly.

In some embodiments, the second tube comprises a throttle device, and the throttle device is placed between the first heat exchanger and the condenser.

In some embodiments, the condenser comprises a heat conduction tube; and wherein the heat conduction tube has one end extending out of the condenser to be connected to the throttle device and another end extending out of the condenser to be connected to the compressor.

In some embodiments, further comprising an air pump, a two-way solenoid valve, and an air tube; wherein: the air pump is connected to the body wrap through the air tube, and the two-way solenoid valve is arranged on the air tube; the air pump and the two-way solenoid valve are both electrically connected to the control assembly; and the body wrap is configured to be inflated by the air pump, and the body wrap is configured to be deflated by the two-way solenoid valve.

In some embodiments, the air tube is further provided with a pressure relief valve and an air pressure sensor, and the air pressure sensor is electrically connected to the control assembly.

In some embodiments, further comprising a shell; wherein the control assembly comprises a main control board, a display screen, and a power interface; the display screen and the power interface are both electrically connected to the main control board; and wherein: the display screen and the power interface are arranged on the outer surface of the shell, the main control board is arranged inside the shell.

In some embodiments, the control assembly comprises a battery, which is arranged inside the shell; the battery is electrically connected to the main control board.

In some embodiments, the shell is further provided with a handle.

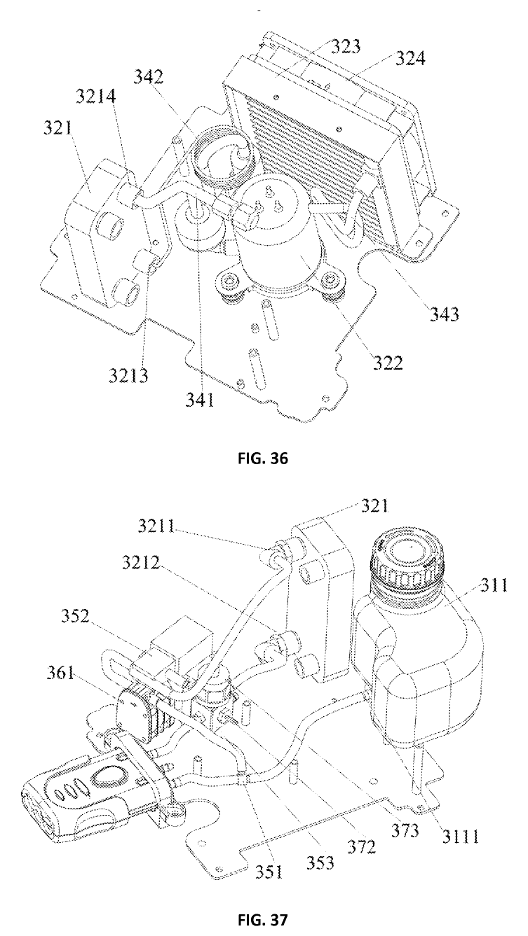

In one embodiment, referring to FIGS. 36 and 37, the cooling component comprises a compressor 322, a condenser 323, and a fan 324, and the control assembly is electrically connected to the compressor 322 and the fan 324, respectively; and wherein: the first heat exchanger 321 comprises an air inlet of the first heat exchanger 3213, an air outlet of the first heat exchanger 3214; the compressor 322 has an air inlet connected to the air outlet of the first heat exchanger 3214 through a first tube 341; the air inlet of the first heat exchanger 3213 is connected to the condenser 323 through a second tube 342; the condenser 323 is connected to an air outlet of the compressor 322 through a third tube 343; the first tube 341 and the second tube 342 are connected by fluid, and the second tube 342 and the third tube 343 are connected by fluid, enabling refrigerant in the compressor 322 to pass through the condenser 323 first and then through the first heat exchanger 321 to achieve a cooling effect; the fan 324 has a working surface facing the condenser 323.

It should be noted that a branch of the first tube 341 is provided with a refrigerant adding port, so as to maintain temperature of the cooling when the cooling mechanism works for a long time. When using the cooling mechanism in long-term circles, it may not achieve the heat exchange in an ideal state and the cooling effect cannot reach the expectation, thus the refrigerant is periodically added to improve the cooling effect.

It should be noted that the first tube 341 and the second tube 342 are connected by fluid, and the second tube 342 and the third tube 343 are connected by fluid. In fact, the first tube 341, the second tube 342 and the third tube 343 are all part of the same tube, and the same tube is connected to the compressor 322, the first heat exchanger 321 and the condenser 323 to form a closed circulation loop. For example, the first tube 341 includes a compressor section, a first tube connecting section extending out of the compressor 322 to connect the first heat exchanger 321, and a first heat exchanger section. The second tube 342 includes a second tube connecting section extending out of the first heat exchanger 321 to connect the condenser 323, and a condenser section. The third tube 343 includes a third tube connecting section extending out of the condenser 323 to connect the compressor 322.

It should be noted that the refrigerant flows into the first heat exchanger 321 through the second tube 342, the air inlet of the first heat exchanger 3213, and then returns to the compressor 322 through the air outlet of the first heat exchanger 3214 and the first tube 341 to cool the first heat exchanger 321 and exchange heat with the liquid in the first heat exchanger 321, so that the temperature of the liquid decreases.

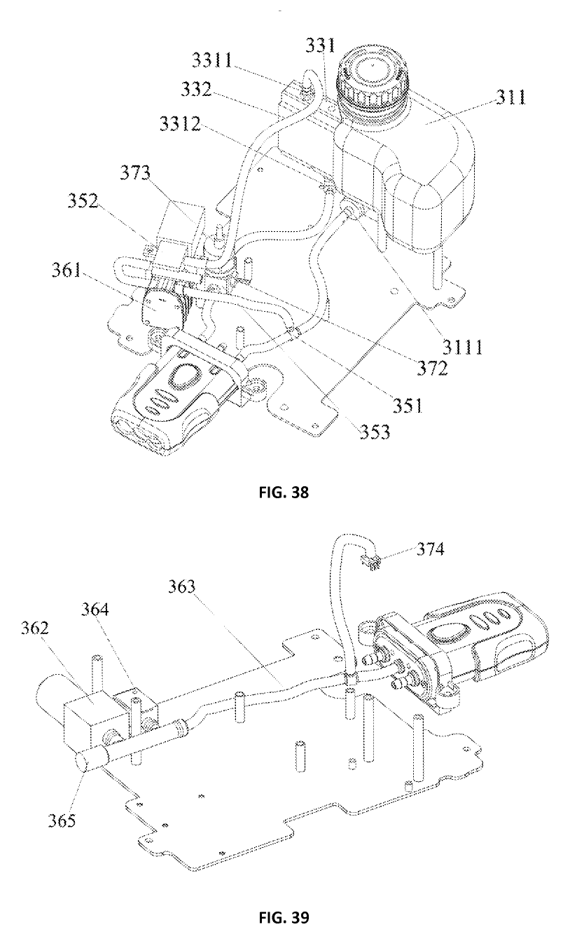

In one embodiment, referring to FIG. 38, the heating component comprises one or more heating sheets 332, which are arranged on the outside of the second heat exchanger 331; the one or more heating sheets 332 are electrically connected to the control assembly.

It should be noted that the heating plate 332 is in contact with the second heat exchanger 331 to heat the second heat exchanger 331 and exchange heat with the liquid in the second heat exchanger 331, so that temperature of the liquid rises.

In one embodiment, further comprising a insulation device, the insulation device is arranged on the outside of the heating sheet 332. The insulation device may be a mica sheet.

In one embodiment, referring to FIG. 37 and FIG. 38 and FIG. 41, further comprising a first multiple-way connector 351, wherein the first multiple-way connector 351 comprises a first connector 3511, a second connector 3512, and a third connector 3513; wherein the first connector 3511 is connected to the liquid outlet of the reservoir 3111, the second connector 3512 is connected to the liquid inlet of the first heat exchanger 3211 and the liquid inlet of the second heat exchanger 3311, the third connector 3513 is connected to the liquid outlet of the body wrap A302.

In one embodiment, referring to FIG. 37 and FIG. 38 and FIG. 41, further comprising a multiple-way solenoid valve 352, which is electrically connected to the control assembly; wherein the multiple-way solenoid valve 352 comprises a fourth connector 3521, a fifth connector 3522, and a sixth connector 3523; wherein the fourth connector 3521 is connected to the second connector 3512, the fifth connector 3522 is connected to the liquid inlet of the first heat exchanger 3211, the sixth connector 3523 is connected to the liquid inlet of the second heat exchanger 3311.

It should be noted that the control assembly can control the fourth connector 3521 to connect with the connector 3522, or the fourth connector 3521 to connect with the sixth connector 3523.

In one embodiment, referring to FIG. 37 and FIG. 38 and FIG. 41, further comprising a second multiple-way connector 353, wherein the second multiple-way connector 353 comprises a seventh connector 3531, a eighth connector 3532, and a ninth connector 3533; wherein the seventh connector 3531 is connected to the liquid outlet of the first heat exchanger 3212, the eighth connector 3532 is connected to the liquid outlet of the second heat exchanger 3312, the ninth connector 3533 is connected to the liquid inlet of the body wrap A301.

In one embodiment, referring to FIG. 37 and FIG. 38 and FIG. 41, further comprising a water pump 361 is arranged on a tube connected between the second connector 3512 and the fourth connector 3521, and the water pump 361 is electrically connected to the control assembly; and wherein, when the fourth connector 3521 and the fifth connector 3522 are connected, in response to the water pump 361 working, the liquid in the reservoir 311 flows into the first heat exchanger 321 through the first multiple-way connector 351 and the multiple-way solenoid valve 352, and then flows into the body wrap A30 through the second multiple-way connector 353; the liquid in the body wrap A30 flows into the first heat exchanger 321 through the first multiple-way connector 351 and the multiple-way solenoid valve 352, and then flows into the body wrap A30 again through the second multiple-way connector 353; when the fourth connector 3521 and the sixth connector 3523 are connected, in response to the water pump 361 working, the liquid in the reservoir 311 flows into the second heat exchanger 331 through the first multiple-way connector 351 and the multiple-way solenoid valve 352, and then flows into the body wrap A30 through the second multiple-way connector 353; the liquid in the body wrap A30 flows into the second heat exchanger 331 through the first multiple-way connector 351 and the multiple-way solenoid valve 352, and then flows into the body wrap A30 again through the second multiple-way connector 353; in response to the water pump 361 stopped working, the liquid in the body wrap A30 flows back to the reservoir 311 through the first multiple-way connector 351.

It should be noted that when the water pump 361 is closed, due to the high pressure in the body wrap A30, the liquid in the body wrap A30 will flow into the reservoir 311 through the first multiple-way connector 351.

In one embodiment, referring to FIG. 41, the reservoir 361 is further provided with a liquid level detector 371, and the liquid level detector 371 is electrically connected to the control assembly.

In one embodiment, referring to FIG. 37 and FIG. 38 and FIG. 41, the second multiple-way connector 353 further comprises a tenth connector 3534, the tenth connector 3534 is connected with a temperature sensor 372, and the temperature sensor 372 is electrically connected to the control assembly.

In one embodiment, referring to FIG. 37 and FIG. 38 and FIG. 41, the second multiple-way connector 353 further comprises a eleventh connector 3535, the eleventh connector 3535 is connected with a water pressure sensor 373, and the water pressure sensor 373 is electrically connected to the control assembly.

It should be noted that the liquid level detector 371 is used to detect the height of a liquid position. The temperature sensor 372 is used to detect the temperature of the liquid flows into the body wrap A30 or flows out the body wrap A30, to control the temperature by means of the control assembly to achieve a suitable cold or hot compression effect. The water pressure sensor 373 is used to detect water pressure and prevent crossing the threshold.

In one embodiment, referring to FIG. 36, the second tube 342 comprises a throttle device, and the throttle device is placed between the first heat exchanger 321 and the condenser 323.

It should be noted that the second tube 342 includes a second tube connecting section extending out of the first heat exchanger 321 to connect the condenser 323, and a condenser section. The throttle device is placed on the second tube connecting section extending out of the first heat exchanger 321 to connect the condenser 323. The throttle device may be a capillary tube section, the second tube connecting section extends out of the first heat exchanger 321 with a tendency of reduced tube diameter to form the capillary tube section. Preferably, the diameter is reduced to 3-6 times the diameter of the original tube (i.e., the second tube connecting section). The throttle device may also be a throttle valve or other throttle step-down device.

It should be noted that, after the refrigerant liquid passes through the capillary tube section with a small diameter, a flowing speed of the refrigerant liquid becomes slow due to a small sectional area of the capillary tube section, so that pressure of the refrigerant liquid is reduced, and meanwhile, the temperature of the refrigerant liquid is reduced. At this point, high-temperature and high-pressure refrigerant liquid just output from the condenser 323 is able to be depressurized through the capillary tube section.

In one embodiment, referring to FIG. 36, the condenser 323 comprises a heat conduction tube; and wherein the heat conduction tube has one end extending out of the condenser 323 to be connected to the throttle device and another end extending out of the condenser 323 to be connected to the compressor 322.

It should be noted that the heat conduction tube includes a copper tube. High temperature and high-pressure refrigerant gas enters the condenser section, and the condenser section is provided with a copper tube. Heat of the refrigerant gas is absorbed and gradually dissipated into surrounding environment by using the copper tube. Meanwhile, the working surface of the fan 324 faces a position of the copper tube in the condenser 323, further makes the refrigerant cooling liquid. The refrigerant liquid subjected to heat dissipation through the copper tube and the fan 324 is conveyed back to the first heat exchanger 321 through the second tube 342.

In one embodiment, referring to FIG. 39, further comprising an air pump 362, a two-way solenoid valve 364, and an air tube 363; wherein: the air pump 362 is connected to the body wrap A30 through the air tube 363, and the two-way solenoid valve 364 is arranged on the air tube 363; the air pump 362 and the two-way solenoid valve 364 are both electrically connected to the control assembly; and the body wrap A30 is configured to be inflated by the air pump 362, and the body wrap A30 is configured to be deflated by the two-way solenoid valve 364.

On the one hand, the body wrap A30 is expanded by inflation, allowing the body wrap A30 to conform more closely to therapy areas, thereby enhancing the therapy effect of the cold therapy with optional compression therapy. On the other hand, the combination of inflation and deflation allows the body wrap A30 to provide medical effects such as massage and compression.

In one embodiment, referring to FIG. 39, the air tube is further provided with a pressure relief valve 365 and an air pressure sensor 374, and the air pressure sensor 374 is electrically connected to the control assembly.

It should be noted that, when the air pressure sensor 374 detects excessive pressure in the trachea, the air pump 362 can be controlled to close and the two-way solenoid valve 364 can be opened to deflate the body wrap A30. When the air pump 362 or the two-way solenoid valve 364 fails, the air can be discharged through the pressure relief valve 365.

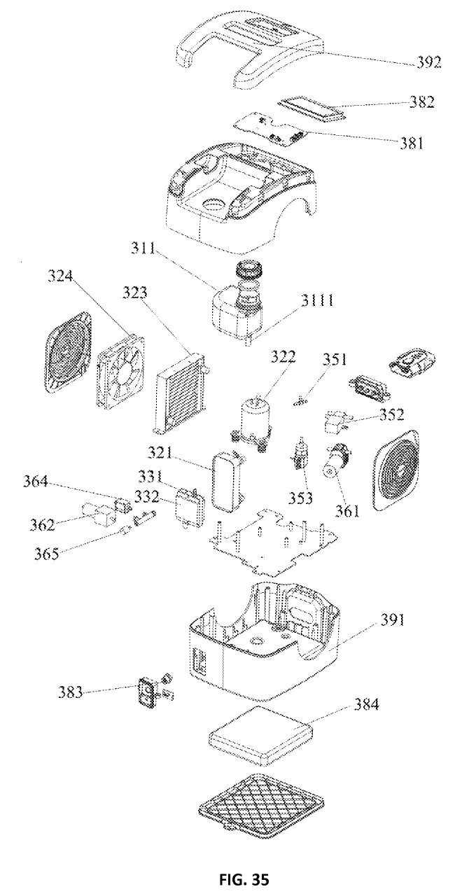

In one embodiment, referring to FIG. 34 and FIG. 35, further comprising a shell 391; wherein the control assembly comprises a main control board 381, a display screen 382, and a power interface 383; the display screen 382 and the power interface 383 are both electrically connected to the main control board 381; and wherein: the display screen 382 and the power interface 383 are arranged on the outer surface of the shell 391, the main control board 381 is arranged inside the shell 391.

It should be noted that the main control board 381 is controlled through the display screen 382, working temperature of the body wrap A30 is able to be set, air pressure values of the body wrap A30 is able to be set, to achieve the effect of cold compress, and the pressing effect is achieved by adjusting the inflation and deflation. The device can be supplied with power through the power interface 383.

In one embodiment, referring to FIG. 35, the control assembly comprises a battery 384, which is arranged inside the shell 391; the battery 384 is electrically connected to the main control board 381.

In one embodiment, referring to FIG. 34 and FIG. 35, the shell 391 is further provided with a handle 392, easy for users to carry.

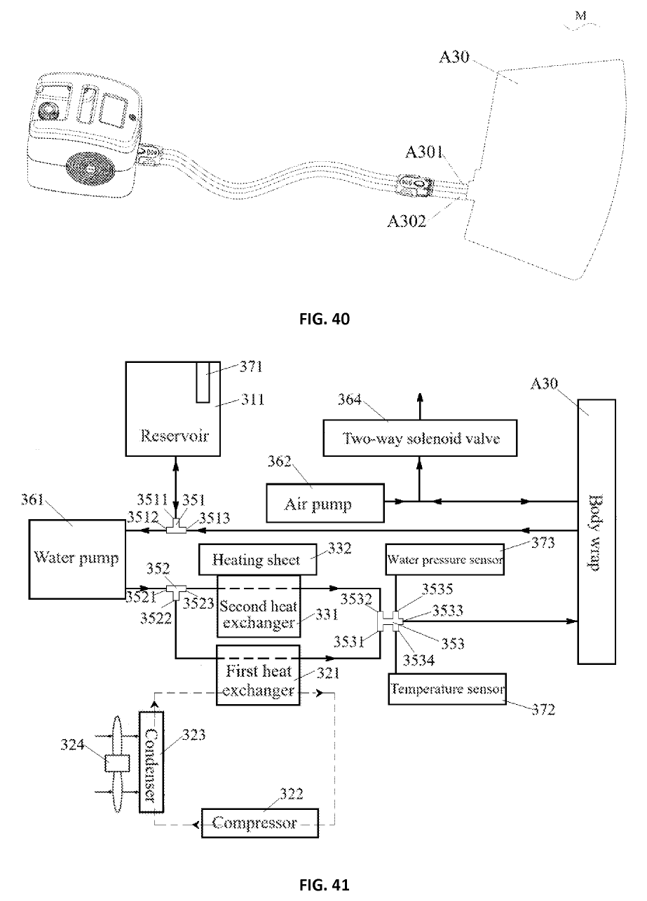

In some examples, referring to FIG. 40 to FIG. 43, the present disclosure provides a portable device for cold therapy with optional heat and compression therapy M including the portable system for cold therapy with optional heat and compression therapy and a body wrap A30. The portable system for cold therapy with optional heat and compression therapy comprising a cooling mechanism, a heating mechanism, a reservoir, and a control assembly; the cooling mechanism comprises a cooling component and a first heat exchanger 321, and the cooling component is used for cooling the first heat exchanger 321; the heating mechanism comprises a heating component and a second heat exchanger 331, and the heating component is used for heating the second heat exchanger 331; the control assembly is electrically connected to the cooling component and the heating component, respectively; the cooling component and the heating component are placed outside the reservoir 311, respectively; and wherein: the reservoir 311 comprises a liquid outlet of the reservoir 3111; the first heat exchanger 321 comprises a liquid inlet of the first heat exchanger 3211, and a liquid outlet of the first heat exchanger 3212; the second heat exchanger 331 comprises a liquid inlet of the second heat exchanger 3311, and a liquid outlet of the second heat exchanger 3312; the liquid in the reservoir 311 can flow into the first heat exchanger 321 through the liquid outlet of the reservoir 3111 and the liquid inlet of the first heat exchanger 3211, or flow into the second heat exchanger 331 through the liquid outlet of the reservoir 3111 and the liquid inlet of the second heat exchanger 3311.

When the liquid in the reservoir 311 flows into the first heat exchanger 321 first, and then flows into the body wrap A30 through the liquid outlet of the first heat exchanger 3212 and the liquid inlet of the body wrap A301; the liquid in the body wrap A30 can flow into the first heat exchanger 321 first through the liquid outlet of the body wrap A302 and the liquid inlet of the first heat exchanger 3211, and then flow into the body wrap A30 again, or the liquid in the body wrap A30 can flow back to the reservoir 311 through the liquid outlet of the body wrap A302 and the liquid outlet of the reservoir 3111.

When the liquid in the reservoir 311 flows into the second heat exchanger 331 first, and then flows into the body wrap A30 through the liquid outlet of the second heat exchanger 3312 and the liquid inlet of the body wrap A301; the liquid in the body wrap A30 can flow into the second heat exchanger 331 first through the liquid outlet of the body wrap A302 and the liquid inlet of the second heat exchanger 3311, and then flow into the body wrap A30 again, or the liquid in the body wrap A30 can flow back to the reservoir 311 through the liquid outlet of the body wrap A302 and the liquid outlet of the reservoir 3111.

The technical problems to be solved in the embodiments lie in insufficient cooling effect, low efficiency and single function of the cooling mechanism.

The portable device for cold therapy with optional heat and compression therapy according to the present disclosure has the following beneficial effects. In some examples, by setting the cooling component and the first heat exchanger, the first heat exchanger 321 can be cooled through the cooling component, the liquid in the reservoir 311 can flow into the body wrap A30 after cooling through the first heat exchanger 321, the liquid in the body wrap A30 can flow into the first heat exchanger 321 again for cooling, or the liquid in the body wrap A30 can flow back into the reservoir 311, providing the body wrap A30 with a cold compression function, and the remaining liquid in the reservoir 311 will no longer participate in the circulation, to greatly improve the cooling efficiency, and reduce the power consumption. Under the condition of the ambient temperature of 25-28℃, referring to FIG. 42, with an area of 1m² of the body wrap A30, a cooling rate is such that the surface temperature of the body wrap A30 reaches 5℃ within about 3 minutes, a cooling power is about 75 watts, and the achieved cooling capacity is about 250 watts. The device exhibits excellent cooling effects, high efficiency, and stability. In some examples, by setting the heating component and the second heat exchanger 331, the second heat exchanger 331 can be heated through the heating component, the liquid in the reservoir 311 can flow into the body wrap A30 after heating through the second heat exchanger 331, the liquid in the body wrap A30 can flow into the second heat exchanger 331 again for heating, or the liquid in the body wrap A30 can flow back into the reservoir 311, providing the body wrap A30 with a hot compression function, and the remaining liquid in the reservoir 311 will no longer participate in the circulation, to greatly improve the heating efficiency, and reduce the power consumption. Under the condition of the ambient temperature of 25-28℃, referring to FIG. 43, with an area of 1m² of the body wrap A30, a heating rate is such that the surface temperature of the body wrap A30 reaches 43℃ within about 5 minutes, a heating power is about 150 watts, and the achieved heating capacity is about 150 watts. The device exhibits excellent heating effects, high efficiency, and stability. The cooling and heating mechanisms are simultaneously incorporated into the portable device for cold therapy with optional heat and compression therapy, provided in the present disclosure. The device has a compact, small, and lightweight structure, facilitating convenient portability.

Furthermore, the heat exchanger of the cooling mechanism and the heating mechanism are set separately, so that the first heat exchanger 321 allows pre-cooling, and the second heat exchanger 331 allows pre-heating, thereby enabling immediate use when the user requires therapy.

In one embodiment, shunt tubes are provided in the body wrap A30.

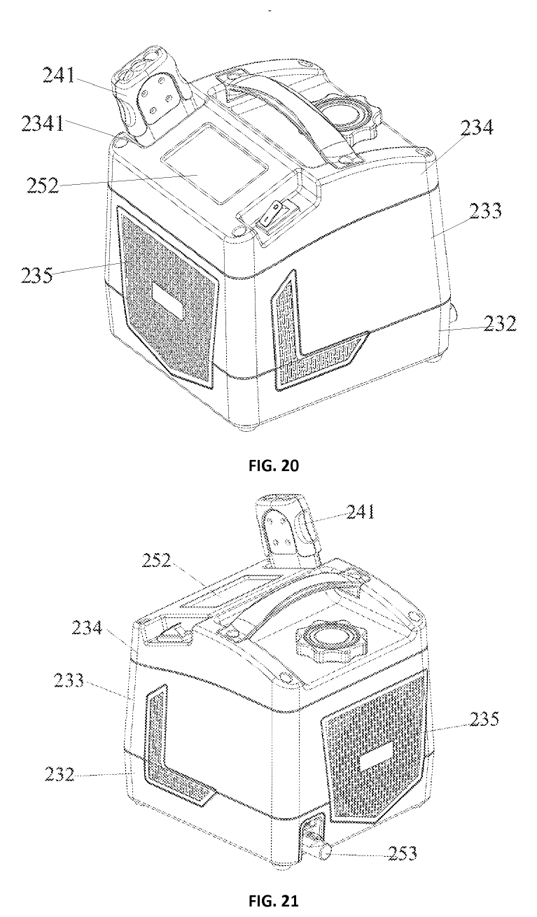

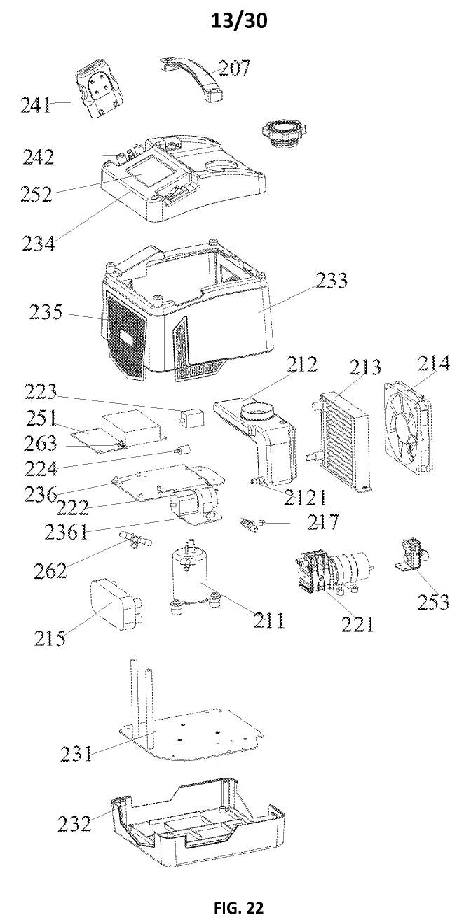

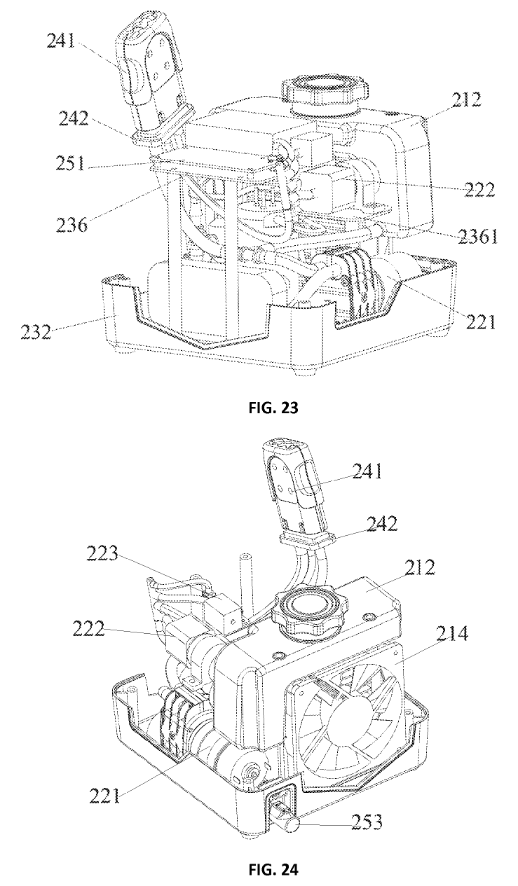

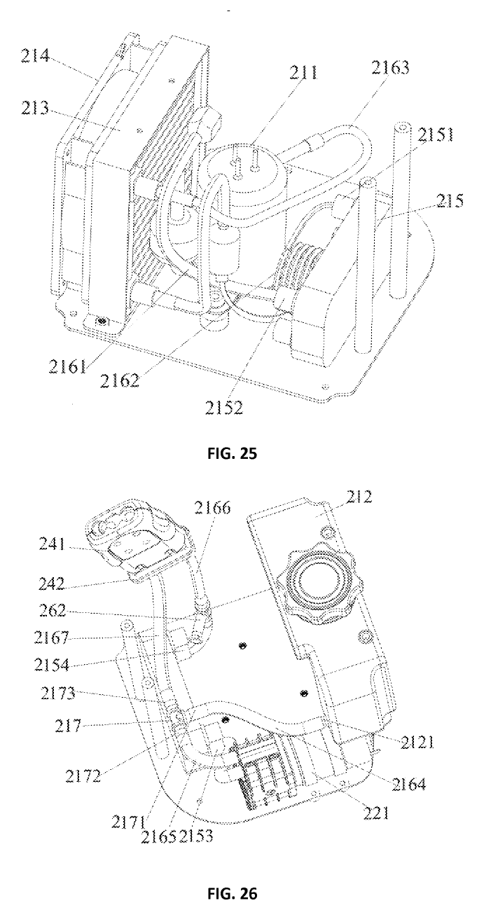

In some examples, referring to FIGS. 20 to 28, the present disclosure provides a portable system for cold therapy with optional compression therapy, applied to a body wrap A20, comprising a cooling mechanism, a reservoir 212, and a control assembly; wherein the cooling mechanism comprises a compressor 211, a condenser 213, a fan 214, and a heat exchanger 215, and the control assembly is electrically connected to the compressor 211, and the fan 214, respectively; and wherein: the compressor 211, the condenser 213, the fan 214, and the heat exchanger 215 are placed outside the reservoir 212, respectively; the compressor 211 has an air inlet connected to the heat exchanger 215 through a first tube 2161; the heat exchanger 215 is connected to the condenser 213 through a second tube 2162; the condenser 213 is connected to an air outlet of the compressor 211 through a third tube 2163; the first tube 2161 and the second tube 2162 are connected by fluid, and the second tube 2162 and the third tube 2163 are connected by fluid; the fan 214 has a working surface facing the condenser 213; and wherein, the reservoir 212 comprises a liquid outlet of the reservoir 2121; the heat exchanger 215 comprises an air inlet of the heat exchanger 2151, an air outlet of the heat exchanger 2152, a liquid inlet of the heat exchanger 2153, and a liquid outlet of the heat exchanger 2154; wherein the air outlet of the heat exchanger 2152 is connected to the first tube 2161, the air inlet of the heat exchanger 2151 is connected to the second tube 2162, the liquid inlet of the heat exchanger 2153 is connected to the liquid outlet of the reservoir 2121, the liquid outlet of the heat exchanger 2154 is connected to a liquid inlet of the body wrap A201, and a liquid outlet of the body wrap A202 is connected to the liquid outlet of the reservoir 2121 and the liquid inlet of the heat exchanger 2153.

Wherein the liquid in the reservoir 212 flows into the heat exchanger 215 first through the liquid outlet of the reservoir 2121 and the liquid inlet of the heat exchanger 2153, and then flows into the body wrap A20 through the liquid outlet of the heat exchanger 2154 and the liquid inlet of the body wrap A201; the liquid in the body wrap A20 can flow into the heat exchanger 215 first through the liquid outlet of the body wrap A202 and the liquid inlet of the heat exchanger 2153, and then flow into the body wrap A20 again, or the liquid in the body wrap A20 can flow back to the reservoir 212 through the liquid outlet of the body wrap A202 and the liquid outlet of the reservoir 2121.

It should be noted that the compressor 211, the condenser 213, the fan 214 and the heat exchanger 215 can all be one or more. A plurality of the compressors 211 are arranged in series. A plurality of the condenser 213 are arranged in series, each working face of the fan 214 is toward the condenser 213. A plurality of the heat exchangers 215 are arranged in series.

It should be noted that the liquid includes water, alcohol, antifreezing solution, and the like. A concentration of 10% alcohol is preferably used.

It should be noted that a branch of the first tube 2161 is provided with a refrigerant adding port, so as to maintain temperature of the cooling when the cooling mechanism works for a long time. When using the cooling mechanism in long-term circles, it may not achieve the heat exchange in an ideal state and the cooling effect cannot reach the expectation, thus the refrigerant is periodically added to improve the cooling effect.

It should be noted that the heat exchanger 215 includes a brazed plate heat exchanger. The liquid in the body wrap A20 flows into the liquid inlet of the heat exchanger 2153 through the liquid outlet of the reservoir 2121, liquid cooled in the brazed plate heat exchanger, and then the liquid flows into the body wrap A20 through the liquid outlet of the heat exchanger 2154.

It should be noted that the first tube 2161 and the second tube 2162 are connected by fluid, and the second tube 2162 and the third tube 2163 are connected by fluid. In fact, the first tube 2161, the second tube 2162 and the third tube 2163 are all part of the same tube, and the same tube is connected to the compressor 211, the heat exchanger 215 and the condenser 213 to form a closed circulation loop. For example, the first tube 2161 includes a compressor section, a first tube connecting section extending out of the compressor 211 to connect the heat exchanger 215, and a heat exchanger section. The second tube 2162 includes a second tube connecting section extending out of the heat exchanger 215 to connect the condenser 213, and a condenser section. The third tube 2163 includes a third tube connecting section extending out of the condenser 213 to connect the compressor 211.

It should be noted that the refrigerant flows into the heat exchanger 215 through the second tube 2162, the air inlet of the heat exchanger 2151, and then returns to the compressor 211 through the air outlet of the heat exchanger 2152 and the first tube 2161 to cool the heat exchanger 215 and exchange heat with the liquid in the heat exchanger 215, so that the temperature of the liquid decreases.

The technical problems to be solved in the embodiments lie in insufficient cooling effect and low efficiency of the cooling mechanism.

The portable system for cold therapy with optional compression therapy according to the present disclosure has the following beneficial effects. That is, the cooling mechanism is equipped with the compressor 211, the condenser 213, the fan 214, and the heat exchanger 215. High temperature and high-pressure refrigerant gas is transported from the compressor 211 to the condenser 213 through the third tube 2163, at this time, the temperature of the refrigerant gas is high, and the fan 214 is utilized to cool the refrigerant gas in the condenser 213, and the refrigerant gas is liquefied. Refrigerant liquid is transported from the condenser 213 to the heat exchanger 215 through the second tube 2162. In the heat exchanger 215, the refrigerant liquid undergoes heat exchange with the liquid in the heat exchanger 215, absorbs heat from the liquid and gasifies, and the liquid temperature decreases. Refrigerant gas is transported from the heat exchanger 215 to the compressor 211 through the first tube 2161, and the compressor 211 compresses the refrigerant gas. The heat exchanger 215 is arranged on the outside of the reservoir 212, the liquid in the reservoir 212 flows into the heat exchanger 215 for cooling through the liquid outlet of the reservoir 2121 and then flows into the body wrap A20. The liquid in the body wrap A20 can flow into the heat exchanger 215 for cooling again, or the liquid in the body wrap A20 can flow back into the reservoir 212, providing the body wrap A20 with a cold compression function, and the remaining liquid in the reservoir 212 will no longer participate in the circulation, to greatly improve the cooling efficiency, and reduce the power consumption. Under the condition of the ambient temperature of 25-28℃, with an area of 1m² of the body wrap A20, a cooling rate is such that the surface temperature of the body wrap A20 reaches 5℃ within about 5 minutes, a cooling power is about 75 watts, and the achieved cooling capacity is about 250 watts. The system exhibits excellent cooling effects, high efficiency, stability, and low power consumption. The device has a compact, small, and lightweight structure, facilitating convenient portability.

In some embodiments, further comprising a three-way connector, wherein the three-way connector comprises a first connector, a second connector, and a third connector; wherein the first connector is connected to the liquid outlet of the reservoir, the second connector is connected to the liquid inlet of the heat exchanger, the third connector is connected to the liquid outlet of the body wrap.

In some embodiments, the liquid outlet of the reservoir is connected to the first connector through a fourth tube, the liquid inlet of the heat exchanger is connected to the second connector through a fifth tube, the liquid outlet of the heat exchanger is connected to the liquid inlet of the body wrap through a sixth tube, the liquid outlet of the body wrap is connected to the third connector through a seventh tube; wherein the fifth tube is provided with a water pump, and the water pump is electrically connected to the control assembly; and wherein, in response to the water pump working, the liquid in the reservoir flows into the heat exchanger through the fourth tube and the fifth tube, and then flows into the body wrap through the sixth tube; the liquid in the body wrap flows into the heat exchanger through the seventh tube and the fifth tube, and then flows into the body wrap again through the sixth tube; in response to the water pump stopped working, the liquid in the body wrap flows back to the reservoir through the seventh tube and the fourth tube.

In some embodiments, the reservoir is further provided with a liquid level detector, and the liquid level detector is electrically connected to the control assembly.

In some embodiments, the sixth tube or the seventh tube is provided with a temperature sensor, and the temperature sensor is electrically connected to the control assembly.

In some embodiments, the second tube comprises a throttle device, and the throttle device is placed between the heat exchanger and the condenser.

In some embodiments, the condenser comprises a heat conduction tube; and wherein the heat conduction tube has one end extending out of the condenser to be connected to the throttle device and another end extending out of the condenser to be connected to the compressor.

In some embodiments, further comprising an air pump, a solenoid valve, and an air tube; wherein: the air pump is connected to the body wrap through the air tube, and the solenoid valve is arranged on the air tube; the air pump and the solenoid valve are both electrically connected to the control assembly; and the body wrap is configured to be inflated by the air pump, and the body wrap is configured to be deflated by the solenoid valve.

In some embodiments, the air tube is further provided with a pressure relief valve and a pressure sensor, and the pressure sensor is electrically connected to the control assembly.

In some embodiments, further comprising a housing, a bracket, and a connector, wherein the housing comprises a bottom shell, an upper shell and a surface cover, the bracket is embedded in the housing, and the bracket comprises a first bracket and a second bracket, the second bracket arranged above the first bracket; and wherein: the compressor, the condenser, the fan, the heat exchanger, and the water pump are both placed on the first bracket; The second bracket is provided with a first groove, and the air pump is arranged on the first groove; an exterior side of the surface cover is provided with a second groove, wherein the connector has one end inserted into the second groove and respectively connected to the sixth tube, the seventh tube, and the air tube, and has another end configured to be connected to a plurality of tubes, enabling the sixth tube, the seventh tube and the air tube to be respectively connected to the body wrap.

In some embodiments, the control assembly further comprises a main control board, a display screen, and a power interface; and wherein: the display screen and the power interface are both electrically connected to the main control board; the display screen is placed on the surface cover, the main control board is arranged on the second bracket, and the power interface is arranged on the bottom shell.

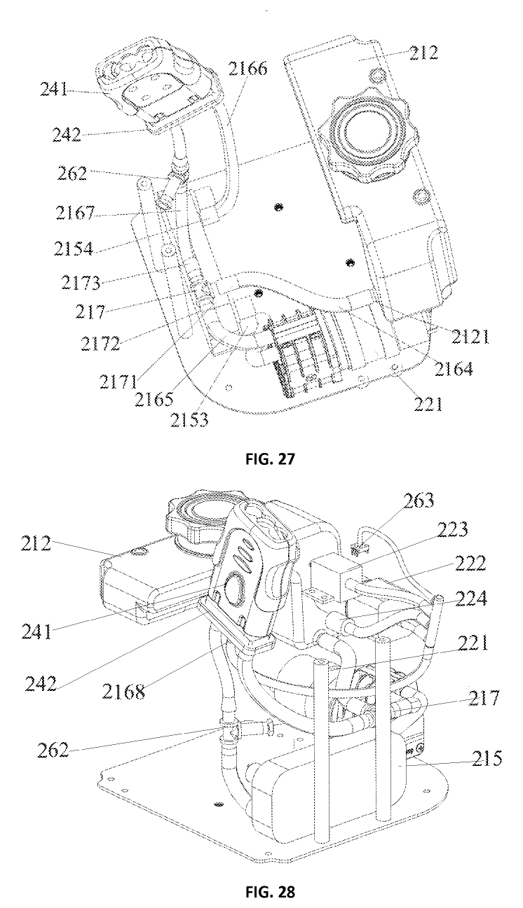

In one embodiment, referring to FIGS. 26 and 27, further comprising a three-way connector 217, wherein the three-way connector 217 comprises a first connector 2171, a second connector 2172, and a third connector 2173; wherein the first connector 2171 is connected to the liquid outlet of the reservoir 2121, the second connector 2172 is connected to the liquid inlet of the heat exchanger 2153, the third connector 2173 is connected to the liquid outlet of the body wrap A202.

In one embodiment, referring to FIG. 26 and FIG. 27, the liquid outlet of the reservoir 2121 is connected to the first connector 2171 through a fourth tube 2164, the liquid inlet of the heat exchanger 2153 is connected to the second connector 2172 through a fifth tube 2165, the liquid outlet of the heat exchanger 2154 is connected to the liquid inlet of the body wrap A201 through a sixth tube 2166, the liquid outlet of the body wrap A202 is connected to the third connector 2173 through a seventh tube 2167; wherein the fifth tube 2165 is provided with a water pump 221, and the water pump 221 is electrically connected to the control assembly; and wherein, in response to the water pump 221 working, the liquid in the reservoir 212 flows into the heat exchanger 215 through the fourth tube 2164 and the fifth tube 2165, and then flows into the body wrap A20 through the sixth tube 2166; the liquid in the body wrap A20 flows into the heat exchanger 215 through the seventh tube 2167 and the fifth tube 2165, and then flows into the body wrap A20 again through the sixth tube 2166; in response to the water pump 221 stopped working, the liquid in the body wrap A20 flows back to the reservoir 212 through the seventh tube 2167 and the fourth tube 2164.

It should be noted that when the water pump 221 is closed, due to the high pressure in the body wrap A20, the liquid in the body wrap A20 will flow into the reservoir 212 through the seventh tube 2167 and the fourth tube 2164.

In one embodiment, the reservoir 212 is further provided with a liquid level detector 261, and the liquid level detector2 61 is electrically connected to the control assembly.

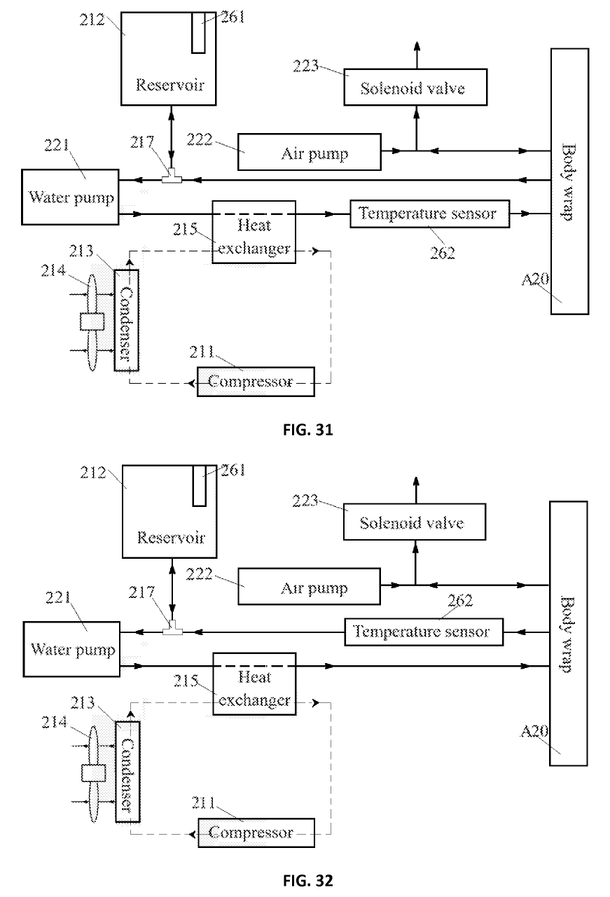

In one embodiment, referring to FIGS. 26 and 31, the sixth tube 2166 is provided with a temperature sensor 262, and the temperature sensor 262 is electrically connected to the control assembly.

In one embodiment, referring to FIG. 27 and FIG. 32, the seventh tube 2167 is provided with a temperature sensor 262, and the temperature sensor 262 is electrically connected to the control assembly.

It should be noted that the liquid level detector 261 is placed in the reservoir 212 for detecting a height of a liquid position. The temperature sensor 262 is placed on the sixth tube 2166 or the seventh tube 2167 for detecting temperature of the liquid flows into the body wrap A20 or flows out the body wrap A20, to control the temperature by means of the control assembly to achieve a suitable cold compression effect.

In one embodiment, the second tube 2162 comprises a throttle device, and the throttle device is placed between the heat exchanger 215 and the condenser 213.

It should be noted that the second tube 2162 includes a second tube connecting section extending out of the heat exchanger 215 to connect the condenser 213, and a condenser section. The throttle device is placed on the second tube connecting section extending out of the heat exchanger 215 to connect the condenser 213. The throttle device may be a capillary tube section, the second tube connecting section extends out of the heat exchanger 215 with a tendency of reduced tube diameter to form the capillary tube section. Preferably, the diameter is reduced to 3-6 times the diameter of the original tube (i.e., the second tube connecting section). The throttle device may also be a throttle valve or other throttle step-down device.

It should be noted that, after the refrigerant liquid passes through the capillary tube section with a small diameter, a flowing speed of the refrigerant liquid becomes slow due to a small sectional area of the capillary tube section, so that pressure of the refrigerant liquid is reduced, and meanwhile, the temperature of the refrigerant liquid is reduced. At this point, high-temperature and high-pressure refrigerant liquid just output from the condenser 213 is able to be depressurized through the capillary tube section.

In one embodiment, the condenser 213 comprises a heat conduction tube; and wherein the heat conduction tube has one end extending out of the condenser 213 to be connected to the throttle device and another end extending out of the condenser 213 to be connected to the compressor 211.

It should be noted that the heat conduction tube includes a copper tube. High temperature and high-pressure refrigerant gas enters the condenser section, and the condenser section is provided with a copper tube. Heat of the refrigerant gas is absorbed and gradually dissipated into surrounding environment by using the copper tube. Meanwhile, the working surface of the fan 214 faces a position of the copper tube in the condenser 213, further makes the refrigerant cooling liquid. The refrigerant liquid subjected to heat dissipation through the copper tube and the fan 214 is conveyed back to the compressor 211 through the third tube 2163.

In one embodiment, further comprising an air pump 222, a solenoid valve 223, and an air tube 2168; wherein: the air pump 222 is connected to the body wrap A20 through the air tube 2168, and the solenoid valve 223 is arranged on the air tube 2168; the air pump 222 and the solenoid valve 223 are both electrically connected to the control assembly; and the body wrap A20 is configured to be inflated by the air pump 222, and the body wrap A20 is configured to be deflated by the solenoid valve 223.

It should be noted that, referring to FIG. 28, the air entering the air tube 2164 is pressurized by the air pump 222 to be conveyed into the body wrap A20, so that the body wrap A20 is inflated. In addition, the solenoid valve 223 is a two-way solenoid valve, the body wrap A20 is deflated by the solenoid valve 223. On the one hand, the body wrap A20 is expanded by inflation, allowing the body wrap A20 to conform more closely to therapy areas, thereby enhancing the therapy effect of the cold therapy with optional compression therapy. On the other hand, the combination of inflation and deflation allows the body wrap A20 to provide medical effects such as massage and compression.

In one embodiment, the air tube 2168 is further provided with a pressure relief valve 224 and a pressure sensor 263, and the pressure sensor 263 is electrically connected to the control assembly.

It should be noted that, when the pressure sensor 263 detects excessive pressure in the trachea, the air pump 222 can be controlled to close and the solenoid valve 223 can be opened to deflate the body wrap A. When the air pump 222 or the solenoid valve 223 fails, the air can be discharged through the pressure relief valve 224.

In one embodiment, further comprising a housing, a bracket, and a connector 241, wherein the housing comprises a bottom shell 232, an upper shell 233 and a surface cover 234, the bracket is embedded in the housing, and the bracket comprises a first bracket 231 and a second bracket 236, the second bracket 236 arranged above the first bracket 231; and wherein: the compressor 211, the condenser 213, the fan 214, the heat exchanger 215, and the water pump 221 are both placed on the first bracket 31; The second bracket 236 is provided with a first groove 2361, and the air pump 222 is arranged on the first groove 2361; an exterior side of the surface cover 234 is provided with a second groove 2341, wherein the connector 241 has one end inserted into the second groove 2341 and respectively connected to the sixth tube 2166, the seventh tube 2167, and the air tube 168, and has another end configured to be connected to a plurality of tubes, enabling the sixth tube 2166, the seventh tube 2167 and the air tube 2168 to be respectively connected to the body wrap A20.

It should be noted that a dust cover 235 is provided at a position, relative to the fan 214, on the side of the upper shell 233 to prevent dust and dirt from entering the interior of the fan 214. A connector base 242 is placed at the second groove 2341, and the connector base 242 is configured to detachably connect the connector 241 to the surface cover 234. The top of the surface cover 234 is also provided with a handle 207, which is convenient to carry.

In one embodiment, the control assembly further comprises a main control board 251, a display screen 252, and a power interface 253; and wherein: the display screen 252 and the power interface 253 are both electrically connected to the main control board 251; the display screen 252 is placed on the surface cover 234, the main control board 251 is arranged on the second bracket 236, and the power interface 253 is arranged on the bottom shell 232.

It should be noted that the main control board 251 is controlled through the display screen 252, working temperature of the body wrap A20 is able to be set, air pressure values of the body wrap A20 is able to be set, to achieve the effect of cold compress, and the pressing effect is achieved by adjusting the inflation and deflation. The device can be supplied with power through the power interface 253.

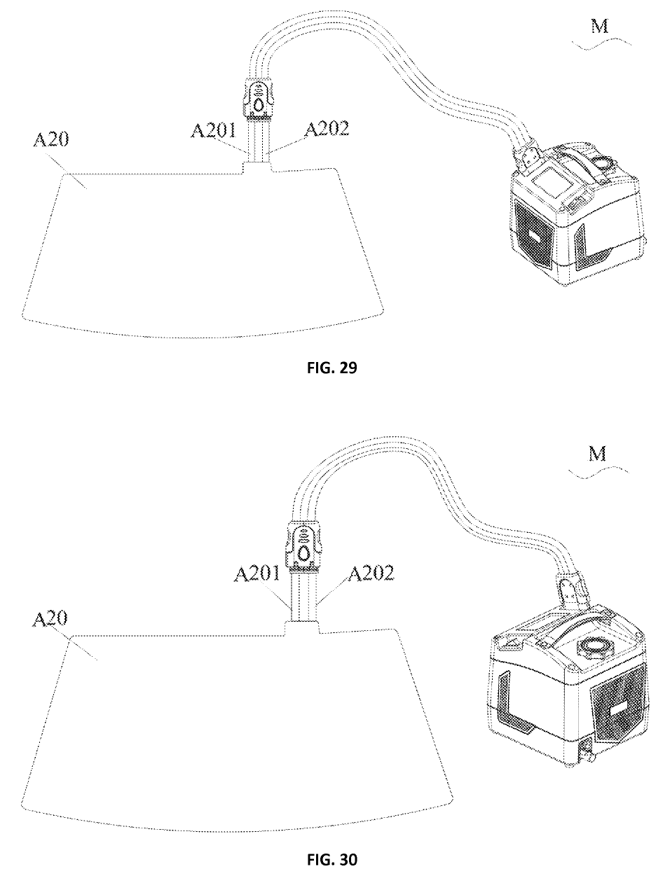

In some examples, referring to FIGS. 29 to 33, the present disclosure provides a portable device for cold therapy with optional compression therapy M including the portable system for cold therapy with optional compression therapy and a body wrap A20. The portable system for cold therapy with optional compression therapy comprising a cooling mechanism, a reservoir 212, and a control assembly; wherein the cooling mechanism comprises a compressor 211, a condenser 213, a fan 214, and a heat exchanger 215, and the control assembly is electrically connected to the compressor 211, and the fan 214, respectively; and wherein: the compressor 211, the condenser 213, the fan 214, and the heat exchanger 215 are placed outside the reservoir 212, respectively; the compressor 211 has an air inlet connected to the heat exchanger 15 through a first tube 161; the heat exchanger 215 is connected to the condenser 213 through a second tube 2162; the condenser 213 is connected to an air outlet of the compressor 211 through a third tube 2163; the first tube 2161 and the second tube 2162 are connected by fluid, and the second tube 2162 and the third tube 2163 are connected by fluid; the fan 214 has a working surface facing the condenser 213; and wherein, the reservoir 212 comprises a liquid outlet of the reservoir 2121; the heat exchanger 215 comprises an air inlet of the heat exchanger 2151, an air outlet of the heat exchanger 2152, a liquid inlet of the heat exchanger 2153, and a liquid outlet of the heat exchanger 2154; wherein the air outlet of the heat exchanger 2152 is connected to the first tube 2161, the air inlet of the heat exchanger 2151 is connected to the second tube 2162, the liquid inlet of the heat exchanger 2153 is connected to the liquid outlet of the reservoir 2121, the liquid outlet of the heat exchanger 2154 is connected to a liquid inlet of the body wrap A201, and a liquid outlet of the body wrap A202 is connected to the liquid outlet of the reservoir 2121 and the liquid inlet of the heat exchanger 2153.

Wherein the liquid in the reservoir 212 flows into the heat exchanger 215 first through the liquid outlet of the reservoir 2121 and the liquid inlet of the heat exchanger 2153, and then flows into the body wrap A20 through the liquid outlet of the heat exchanger 2154 and the liquid inlet of the body wrap A201; the liquid in the body wrap A20 can flow into the heat exchanger 215 first through the liquid outlet of the body wrap A202 and the liquid inlet of the heat exchanger 2153, and then flow into the body wrap A20 again, or the liquid in the body wrap A20 can flow back to the reservoir 212 through the liquid outlet of the body wrap A202 and the liquid outlet of the reservoir 2121.

The technical problems to be solved in the embodiments lie in insufficient cooling effect and low efficiency of the cooling mechanism.

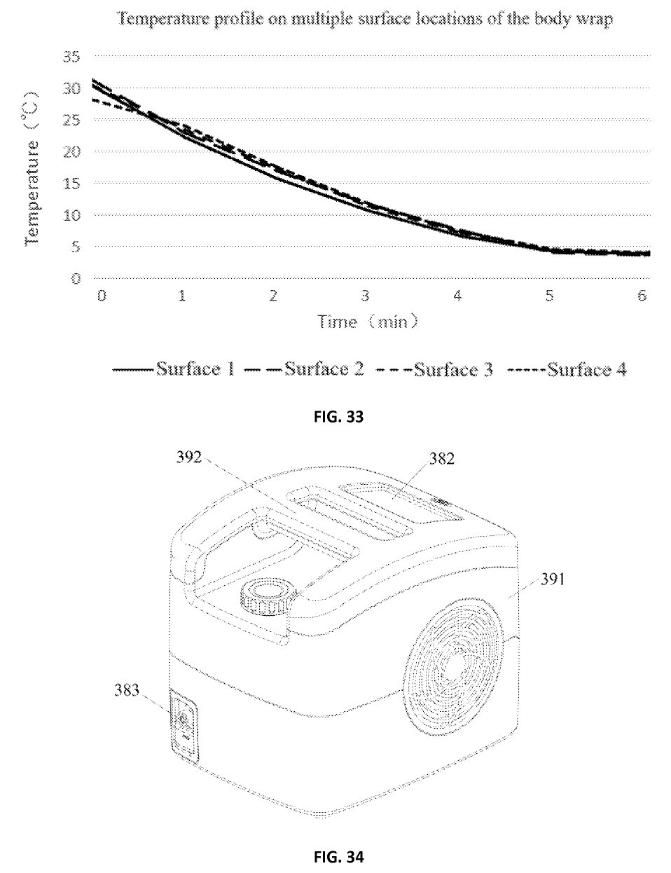

In accordance with the portable device for cold therapy with optional compression therapy M provided in the present disclosure, the advantageous effects include the following. In some examples, the cooling mechanism is equipped with the compressor 211, the condenser 213, the fan 214, and the heat exchanger 215. High temperature and high-pressure refrigerant gas is transported from the compressor 211 to the condenser 213 through the third tube 2163, at this time, the temperature of the refrigerant gas is high, and the fan 214 is utilized to cool the refrigerant gas in the condenser 213, and the refrigerant gas is liquefied. Refrigerant liquid is transported from the condenser 213 to the heat exchanger 215 through the second tube 2162. In the heat exchanger 215, the refrigerant liquid undergoes heat exchange with the liquid in the heat exchanger 215, absorbs heat from the liquid and gasifies, and the liquid temperature decreases. Refrigerant gas is transported from the heat exchanger 215 to the compressor 211 through the first tube 2161, and the compressor 211 compresses the refrigerant gas. The heat exchanger 215 is arranged on the outside of the reservoir 212, the liquid in the reservoir 212 flows into the heat exchanger 215 for cooling through the liquid outlet of the reservoir 2121 and then flows into the body wrap A20. The liquid in the body wrap A20 can flow into the heat exchanger 215 for cooling again, or the liquid in the body wrap A20 can flow back into the reservoir 212, providing the body wrap A20 with a cold compression function, and the remaining liquid in the reservoir 212 will no longer participate in the circulation, to greatly improve the cooling efficiency, and reduce the power consumption. Referring to FIG. 33, under the condition of the ambient temperature of 25-28℃, with an area of 1m² of the body wrap A, a cooling rate is such that the surface temperature of the body wrap A20 reaches 5℃ within about 5 minutes, a cooling power is about 75 watts, and the achieved cooling capacity is about 250 watts. In some examples, the air pumps 22 and the solenoid valves 23 are provided to inflate the body wrap A, allowing the body wrap A20 to conform more closely to therapy areas. This enhances the therapy effect of the cold therapy with optional compression therapy. Additionally, the combination of inflation and deflation allows the body wrap A20 to provide medical effects such as massage and compression. The device has a compact, small, and lightweight structure, facilitating convenient portability. The device is versatile, offering capabilities for cold therapy, and/or massage, enhancing the user experience.

In one embodiment, shunt tubes are provided in the body wrap A20.

new paragraph...

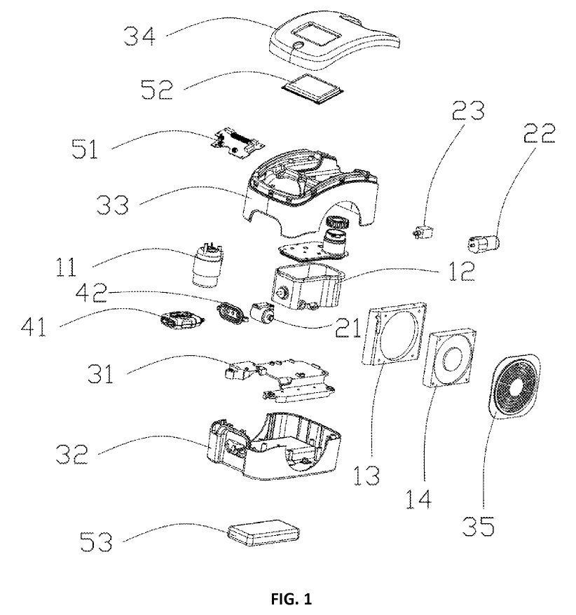

In some examples, referring to FIGS. 1 to 15, the present disclosure provides a portable system for cold therapy with optional heat and compression therapy, applied to a first body wrap A, including a body wrap cooling mechanism, a reservoir 12, and a control assembly. The body wrap cooling mechanism includes a compressor 11, a condenser 13, a fan 14, and a heat exchanger 15. The control assembly is electrically connected to the compressor 11, and the fan 14, respectively. The compressor 11 is placed outside the reservoir 12, and the condenser 13 is respectively placed adjacent to the reservoir 12 and the fan 14. The heat exchanger 15 is placed inside and/or underneath the reservoir 12 and is in direct and/or indirect contact with liquid in the reservoir 12 to cool the liquid. An air outlet of the compressor 11 is connected to the heat exchanger 15 through a first tube 171. The heat exchanger 15 is connected to the condenser 13 through a second tube 172. The condenser 13 is connected to an air inlet of the compressor 11 through a third tube 173. The first tube 171 is communicated with the second tube 172, and the second tube 172 is communicated with the third tube 173. A working surface of the fan 14 faces the condenser 13. The reservoir 12 includes a liquid outlet 122 of the reservoir and a liquid inlet 121 of the reservoir. The liquid in the reservoir 12 flows into the first body wrap A through the liquid outlet 122 of the reservoir, and liquid in the first body wrap A flows into the reservoir 12 through the liquid inlet 121 of the reservoir.

It should be noted that the liquid includes water, alcohol, and the like. Preferably, a concentration of the alcohol is 10%.

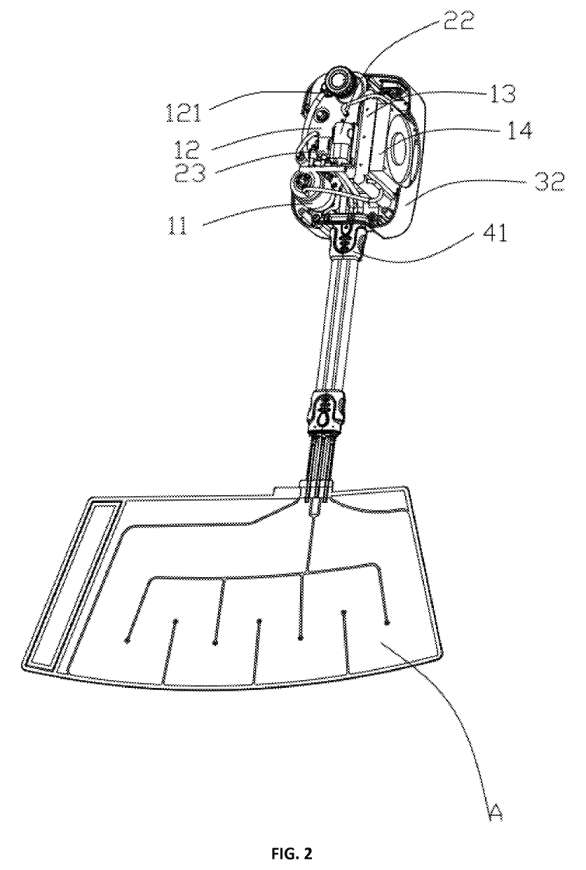

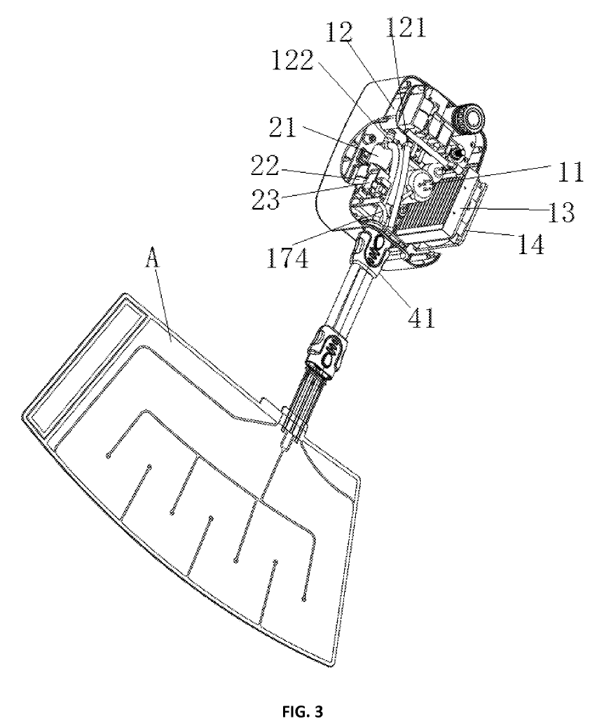

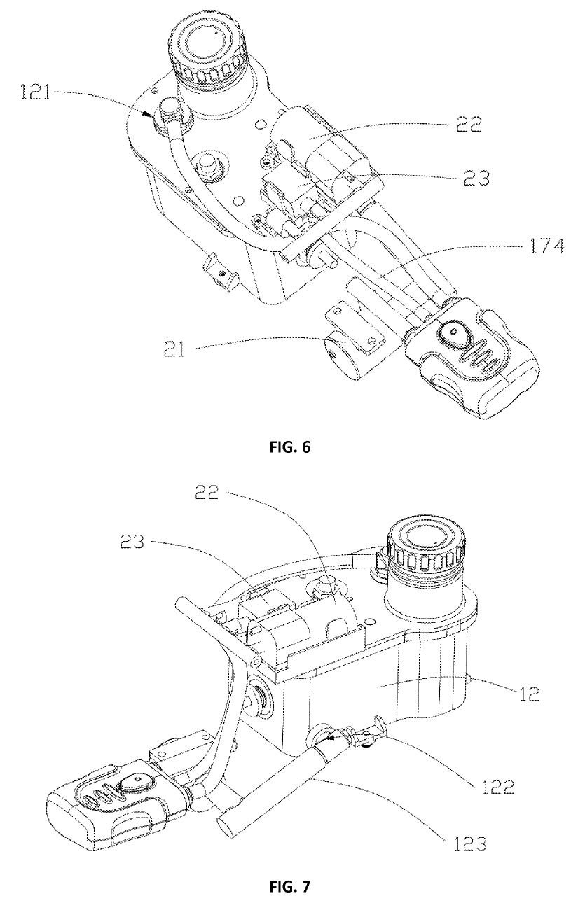

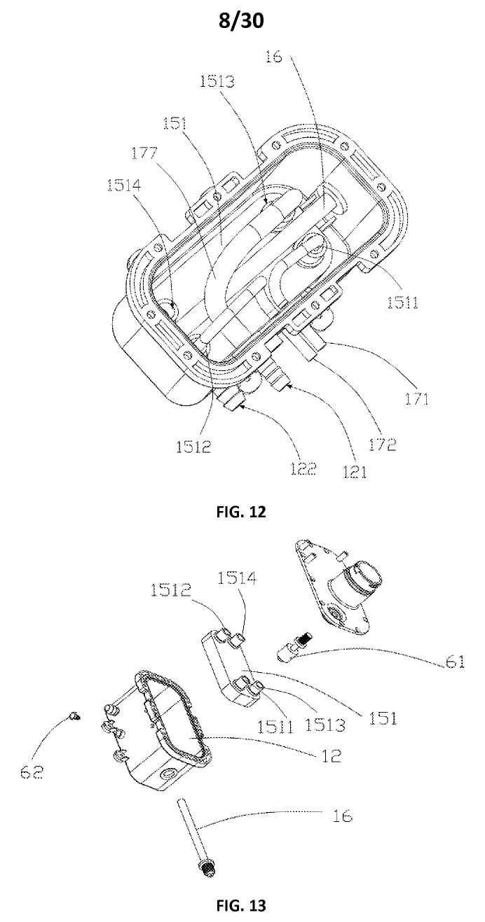

It should be noted that the liquid inlet 121 of the reservoir may be placed at the top of the reservoir as shown in FIGS. 2 and 6, and the liquid inlet 121 of the reservoir may also be placed on the side of the reservoir as shown in FIGS. 3 and 12.

It should be noted that a branch of the first tube 171 is provided with a cooling agent adding port, so as to maintain temperature of the cooling when the body wrap cooling mechanism works for a long time. When using the body wrap cooling mechanism in long-term circles, it may not achieve the heat exchange in an ideal state and the cooling effect cannot reach the expectation, thus the cooling agent is periodically added to improve the cooling effect.

It should be noted that the first tube 171 is communicated with the second tube 172, and the second tube 172 is communicated with the third tube 173. In fact, the first tube 171, the second tube 172 and the third tube 173 are all part of the same tube, and the same tube is connected to the compressor 11, the heat exchanger 15 and the condenser 13 to form a closed circulation loop. For example, the first tube 171 includes a compressor section, a first tube connecting section extending out of the compressor 11 to connect the heat exchanger 15, and a heat exchanger section. The second tube 172 includes a second tube connecting section extending out of the heat exchanger 15 to connect the condenser 13, and a condenser section. The third tube 173 includes a third tube connecting section extending out of the condenser 13 to connect the compressor 11.

The technical problems to be solved in the embodiments lie in insufficient cooling effect and low efficiency of the body wrap cooling mechanism.

The portable system for cold therapy with optional heat and compression therapy according to the present disclosure has the following beneficial effects. That is, the body wrap cooling mechanism is provided with the compressor 11, the condenser 13, the fan 14 and the heat exchanger 15, the liquid gas is transported from the compressor 11 to the heat exchanger 15 through the first tube 171. In the heat exchanger 15, the liquid gas undergoes heat exchange with the liquid in the reservoir 12, absorbs heat from the liquid and gasifies, and the liquid temperature decreases. The gasified gas is then transported from the heat exchanger 15 to the condenser 13 through the second tube 172. At this point, the temperature of the gasified gas transported from the heat exchanger 15 to the condenser 13 is relatively high, and the fan 14 is used to cool the gasified gas in the condenser 13. The cooled gas is then transported from the condenser 13 to the compressor 11 through the third tube 173, and the compressor 11 compresses and liquefies the gas. This process circulates to cool the liquid in the reservoir 12. A cooling rate is such that the liquid decreases from room temperature to 5°C within about 10 minutes. A cooling power is about 75 watts, and the achieved cooling capacity is about 150 watts. The system exhibits excellent cooling effects, high efficiency, stability, and low power. The liquid with decreased temperature in the reservoir 12 flows into the first body wrap A through the liquid outlet 122 of the reservoir, and the liquid in the first body wrap A flows into the reservoir through the liquid inlet 121 of the reservoir, achieving a circulation of liquid and temperature reduction. The first body wrap A is used for cold compression therapy on the body to achieve therapeutic effects. Additionally, an overall volume of the system is approximately 0.014 m³, with a mass of about 3.8 kg, which has a compact, small, and lightweight structure, facilitating convenient portability.

In one embodiment, referring to FIG. 11, the heat exchanger 15 includes a first cooling plate 151, and the first cooling plate 151 is placed inside the reservoir 12. The first cooling plate 151 is provided with a first air inlet 1511 and a first air outlet 1512. The first air inlet 1511 is connected to the first tube 171, and the first air outlet 1512 is connected to the second tube 172.

It should be noted that the liquid gas enters the first cooling plate 151 in the reservoir 12 through the first tube 171 and the first air inlet 1511 and then exits through the first air outlet 1512 and the second tube 172. This process allows the first cooling plate 151 to cool down and undergo heat exchange with the liquid in the reservoir 12, thereby reducing temperature of the liquid in the reservoir 12.

In one embodiment, referring to FIGS. 12 and 13, the first cooling plate 151 is further provided with a liquid inlet 1513 of the first cooling plate and a liquid outlet 1514 of the first cooling plate, and the liquid inlet 1513 of the first cooling plate is connected to the liquid inlet 121 of the reservoir through a tube.

It should be noted that the first cooling plate 151 includes a brazed plate heat exchanger. The liquid in the first body wrap A flows into the liquid inlet 1513 of the first cooling plate through the liquid inlet 121 of the reservoir, and the liquid flows into the reservoir 12 through the liquid outlet 1514 of the first cooling plate after being cooled in the brazed plate heat exchanger.

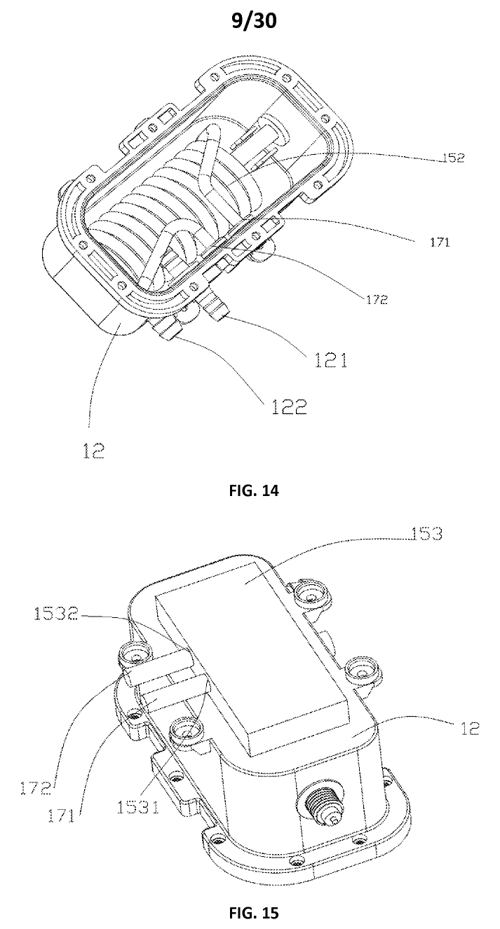

In one embodiment, referring to FIG. 14, the heat exchanger 15 includes a spiral tube 152, and the spiral tube 152 is placed inside the reservoir 12. One end of the spiral tube 152 is connected to the first tube 171, and the other end of the spiral tube 152 is connected to the second tube 172.

It should be noted that the spiral tube 152 includes a copper material or a stainless-steel material. Gas enters the spiral tube 152 in the reservoir 12 through the first tube 171 and then exits through the second tube 172, so that the spiral tube 152 is cooled, and then the temperature of the liquid in the reservoir 12 is reduced through the spiral tube 152. The spiral structure of the spiral tube 152 enables cold gas conveyed by the compressor 11 to stay in the spiral tube 152 for a longer time and increases a contact area between the spiral tube 152 with the liquid in the reservoir 12, so as to perform sufficient heat exchange with the liquid, thereby improving the cooling efficiency during cold therapy.

In one embodiment, referring to FIG. 15, the heat exchanger 15 includes a second cooling plate 153, and the second cooling plate 153 is placed underneath the reservoir 12. The second cooling plate 153 is provided with a second air inlet 1531 and a second air outlet 1532, the second air inlet 1531 is connected to the first tube 171, and the second air outlet 1532 is connected to the second tube 172.The reservoir 12 includes a heat-conducting material.

It should be noted that both the reservoir 12 and the second cooling plate 153 include copper materials. Gas enters the second cooling plate 153 underneath the reservoir 12 through the first tube 171 and the second air inlet 1531 and then exits through the second air outlet 1532 and the second tube 172, so that the second cooling plate 153 is cooled, and the reservoir 12 is integrally cooled through the second cooling plate 153, thereby reducing the temperature of the liquid in the reservoir 12. The second cooling plate 153 and the reservoir 12 may be integrally formed or may be separately placed.

In one embodiment, the heat exchanger 15 includes one or more combinations of the first cooling plate 151, the spiral tube 152, and the second cooling plate 153.

It should be noted that the first cooling plate 151, the spiral tube 152 or the second cooling plate 153 are cooled after the gas enters the first cooling plate 151, the spiral tube 152 or the second cooling plate 153 through the first tube 171, so that the temperature of the liquid in the reservoir 12 is reduced.

In one embodiment, a liquid level detector 61 and a temperature sensor 62 are further provided in the reservoir 12, and the liquid level detector 61 and the temperature sensor 62 are both electrically connected to the control assembly.

It should be noted that the liquid level detector 61 is placed in the reservoir 12 for detecting a height of a liquid position. The temperature sensor 62 is placed on the side of the reservoir 12 for detecting temperature of the liquid in the reservoir 12, to control the temperature by means of the control assembly to achieve a suitable cold compression or hot compression effect.

In one embodiment, the second tube 172 includes a capillary tube section, and the capillary tube section is placed between the heat exchanger 15 and the condenser 13.

It should be noted that the second tube 172 includes a second tube connecting section extending out of the heat exchanger 15 to connect the condenser 13, and a condenser section. The capillary tube section is placed on the second tube connecting section extending out of the heat exchanger 15 to connect the condenser 13. The second tube connecting section extends out of the heat exchanger 15 with a tendency of reduced tube diameter to form the capillary tube section. Preferably, the diameter is reduced to 0.2 times the diameter of the original tube (i.e., the second tube connecting section).

It should be noted that, after the gas used as the cooling agent passes through the capillary tube section with a small diameter, a flowing speed of the gas becomes slow due to a small sectional area of the capillary tube section, so that pressure of the gas is reduced, and meanwhile, the temperature of the gas is reduced. At this point, high-temperature and high-pressure gas just output from the heat exchanger 15 is able to be depressurized through the capillary tube section.

In one embodiment, the condenser 13 includes a heat conduction tube, and the heat conduction tube is U-shaped and is bent along an inner wall of the condenser 13. One end of the heat conduction tube extends out of the condenser 13 to be connected to the capillary tube section, and the other end of the heat conduction tube extends out of the condenser 13 to be connected to the compressor 11.

It should be noted that the heat conduction tube includes a copper tube. The gas enters the condenser section after being depressurized through the capillary tube section, and the condenser section is provided with a copper tube. Heat of the gas is absorbed and gradually dissipated into surrounding environment by using the copper tube. Meanwhile, the working surface of the fan 14 faces a position of the copper tube in the condenser 13, further enhancing heat dissipation of the gas. The gas subjected to heat dissipation through the copper tube and the fan 14 is conveyed back to the compressor 11 through the third tube 173.

In one embodiment, a water pump 21 is further provided, the liquid outlet 122 of the reservoir is connected to a liquid inlet of the first body wrap through the water pump 21, and the water pump 21 is electrically connected to the control assembly. In response to the water pump 21 working, the liquid in the reservoir 12 flows into the first body wrap A through the liquid outlet 122 of the reservoir and the liquid inlet of the first body wrap, and the liquid in the first body wrap A flows back into the reservoir 12 through the liquid outlet of the first body wrap and the liquid inlet 121 of the reservoir.

It should be noted that the liquid inlet 121 of the reservoir is connected to a first adapter, and the liquid outlet 122 of the reservoir is connected to a second adapter 123. The first adapter and the second adapter 123 are respectively configured to connect the liquid tube. The liquid in the reservoir 12 flows out of the liquid outlet 122 of the reservoir and then flows into the first body wrap A after being pressurized by the water pump 21.

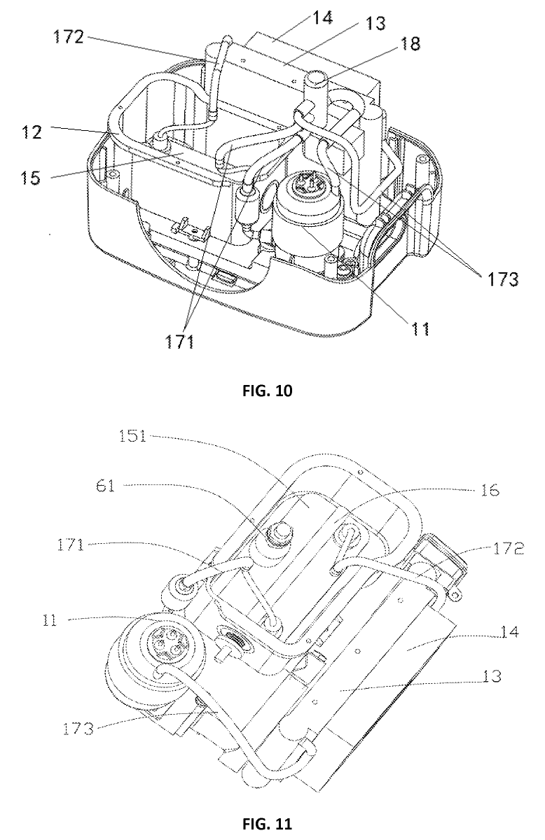

In one embodiment, referring to FIG. 10, a four-way valve 18 is further provided. The compressor 11 is connected to the four-way valve 18 and then to the heat exchanger 15 through the first tube 171. The condenser 13 is connected to the four-way valve 18 and then to the compressor 11 through the third tube 173. The four-way valve 18 is electrically connected to the control assembly, and the four-way valve 18 is controlled by the control assembly to change a gas flow direction, so that the gas in the compressor 11 passes through the condenser 13 first and then through the heat exchanger 15.

It should be noted that the heat exchanger 15 may be the first cooling plate 151, the spiral tube 152, or the second cooling plate 153. The control assembly controls the passage inside the four-way valve 18, causing liquefied gas in the compressor 11 to first pass through the condenser 13. In the condenser 13, the liquefied gas absorbs heat, gasifies, and the temperature of the gas increases. The gas with increased temperature then releases heat as it passes through the heat exchanger 15, subsequently raising the temperature of the liquid inside the reservoir 12. The gas, after being cooled, returns to the compressor 11. By providing the four-way valve 18, only one heat exchanger 15 needs to be provided to cool or heat the liquid, thereby reducing component settings (such as a heating tube 16) and saving costs.

In one embodiment, referring to FIGS. 12 to 14, a body wrap heating mechanism is further provided, and the body wrap heating mechanism includes a heating tube 16. The heating tube 16 is placed inside the reservoir 12 and is electrically connected to the control assembly.

It should be noted that, when the liquid in the reservoir 12 needs to be heated, the compressor 11, the condenser 13 and the fan 14 are controlled to pause by the control assembly, and the heating tube 16 is activated by the control assembly to increase the temperature of the liquid in the reservoir 12. The liquid with increased temperature flows into the first body wrap A through the liquid inlet of the first body wrap, providing the first body wrap A with a hot compression function. The body wrap heating mechanism is controlled by the control assembly to increase the temperature of the liquid in the reservoir 12. A heating rate is such that the liquid reaches 43°C from room temperature within about 6 minutes. A heating power is about 150 watts, and the achieved heating capacity is about 150 watts, thus having excellent heating effects, high efficiency, and stability.

In one embodiment, a first air pump 22, a first solenoid valve 23, and a first air tube 174 are further provided. The first air pump 22 is connected to the first air tube 174 through the first solenoid valve 23, and the first air tube 174 is connected to the first body wrap A. The first air pump 22 and the first solenoid valve 23 are both electrically connected to the control assembly. The first body wrap A is inflated by the first air pump 22, and the first body wrap A is deflated by the first solenoid valve 23.

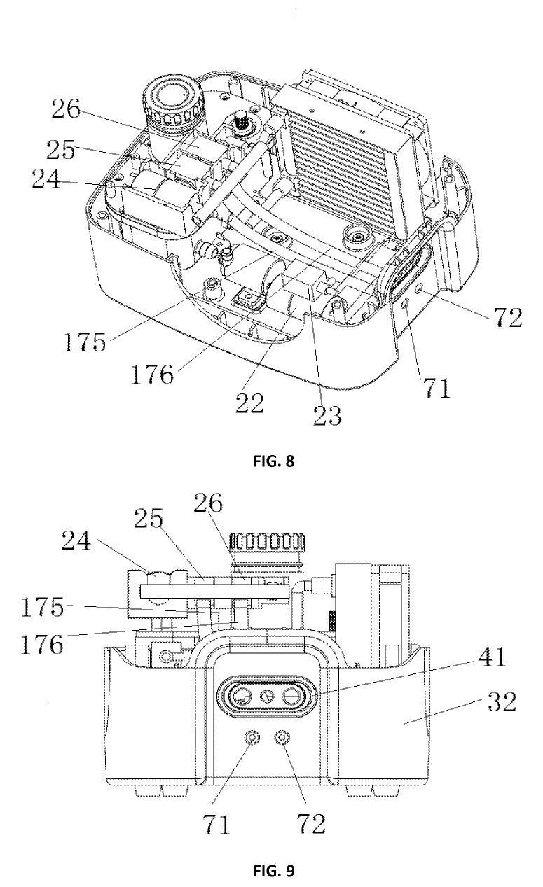

It should be noted that, referring to FIG. 2, FIGS. 6 and 7, the first air pump 22 and the first solenoid valve 23 may be placed at the top of the reservoir 12. Alternatively, referring to FIGS. 3 and 8, the first air pump 22 and the first solenoid valve 23 may be placed in front of the reservoir 12 and located at the side of the compressor 11. The air entering the first air tube 174 is pressurized by the first air pump 22 to be conveyed into the first body wrap A, so that the first body wrap A is inflated. In addition, the first body wrap A is deflated by the first solenoid valve 23. On the one hand, the first body wrap A is expanded by inflation, allowing the first body wrap A to conform more closely to therapy areas, thereby enhancing the therapy effect of the cold therapy with optional heat and compression therapy. On the other hand, the combination of inflation and deflation allows the first body wrap A to provide medical effects such as massage and compression.

In one embodiment, a second air pump 24, a second solenoid valve 25, a third solenoid valve 26, a second air tube 175, and a third air tube 176 are further provided. The second air pump 24 is connected to the second air tube 175 through the second solenoid valve 25, and the second air pump 24 is connected to the third air tube 176 through the third solenoid valve 26. The second air tube 175 is connected to a second body wrap B, and the third air tube 176 is connected to a third body wrap C. The second air pump 24, the second solenoid valve 25, and the third solenoid valve 26 are all electrically connected to the control assembly. The second body wrap B and the third body wrap C are inflated by the second air pump 24, the second body wrap B is deflated by the second solenoid valve 25, and the third body wrap C is deflated by the third solenoid valve 26.

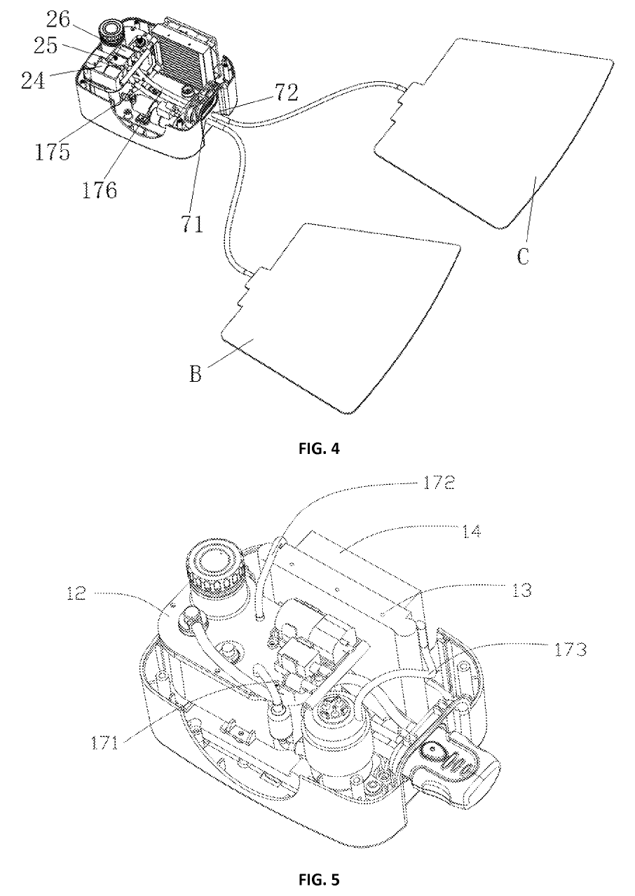

It should be noted that, referring to FIG. 4, FIG. 8, FIG. 9, the second air pump 24, the second solenoid valve 25, and the third solenoid valve 26 are placed at the top of the reservoir 12, and the first air pump 22 and the first solenoid valve 23 are placed in front of the reservoir 12. When a user only needs an air pressure function, the body wraps are communicated with the second air tube 175 and/or the third air tube 176. The air entering the second air tube 175 is pressurized by the second air pump 24 and then conveyed into the second body wrap B, and the air entering the third air tube 176 is pressurized by the second air pump 24 and then conveyed into the third body wrap C, so that the second body wrap B and the third body wrap C are inflated. In addition, the second body wrap B is deflated by the second solenoid valve 25, and the third body wrap C is deflated by the third solenoid valve 26. The combination of inflation and deflation allows the second body wrap B and the third body wrap C to provide medical effects such as massage and compression. The device may simultaneously connect the first body wrap A, the second body wrap B, and the third body wrap C for use by three users at the same time, achieving a high utilization rate of the device.

In one embodiment, a housing, a bracket 31 and a connector 41 are provided. The housing includes a bottom shell 32, an upper shell 33, and a surface cover 34. The bracket 31 is embedded in the housing, and the reservoir 12 is placed on the bracket 31. An avoidance portion is provided on the bracket, and the condenser 13 is embedded in the avoidance portion. A first groove is formed on an exterior side of the bottom shell 32. One end of the connector 41 is inserted into the first groove to respectively connect to the liquid outlet 122 of the reservoir, the liquid inlet 121 of the reservoir and the first air tube 174. The other end of the connector 41 is configured to be connected to a plurality of tubes, enabling the liquid outlet 122 of the reservoir, the liquid inlet 121 of the reservoir and the first air tube 174 to be respectively connected to the first body wrap A.

Referring to FIG. 8 and FIG. 9, the bottom shell 32 is further provided with a first air hole 71 and a second air hole 72, and the first air hole 71 and the second air hole 72 are placed below the first groove. One side of the first air hole 71 is connected to the second air tube 175, and one side of the second air hole 72 is connected to the third air tube 176. The other side of the first air hole 71 and the other side of the second air hole 72 are configured to be connected to tubes, enabling the second air tube 175 to be connected to the second body wrap B and the third air tube 176 to be connected to the third body wrap C.

It should be noted that the bracket 31 is placed at the top of the bottom shell 32 in the housing. The bracket 31 is placed on a side of the bottom shell 32 facing the reservoir 12.

It should be noted that a dust cover 35 is provided at a position, relative to the fan 14, on the side of the housing to prevent dust and dirt from entering the interior of the fan 14.

It should be noted that a connector base 42 is placed at the first groove, and the connector base 42 is configured to detachably connect the connector 41 to the bottom shell 32.

In one embodiment, the control assembly includes a circuit board 51, a display screen 52, and a battery 53. The display screen 52 is electrically connected to the circuit board 51. The display screen 52 is placed on the surface cover 34. An exterior side of the bottom shell 32 facing the bracket 31 is recessed to form a second groove, and the battery 53 is placed in the second groove. The battery includes a storage battery.

It should be noted that the circuit board 51 is controlled through the display screen 52, working temperature of the first body wrap A is able to be set, air pressure values of the first body wrap A, the second body wrap B and the third body wrap C are able to be set, and the pressing effect is achieved by adjusting the inflation and deflation.

It should be noted that the battery is rechargeable, which is convenient in the absence of alternating current. For example, the device in the present disclosure is also able to work normally when outdoors, which is convenient for users to use. Furthermore, the battery is able to be detached from the second groove, which is convenient for charging and storage.

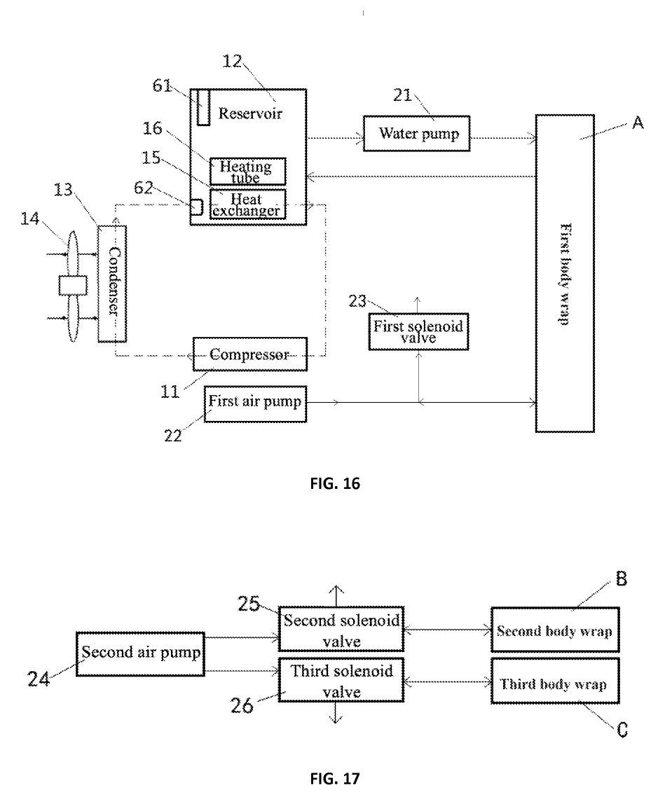

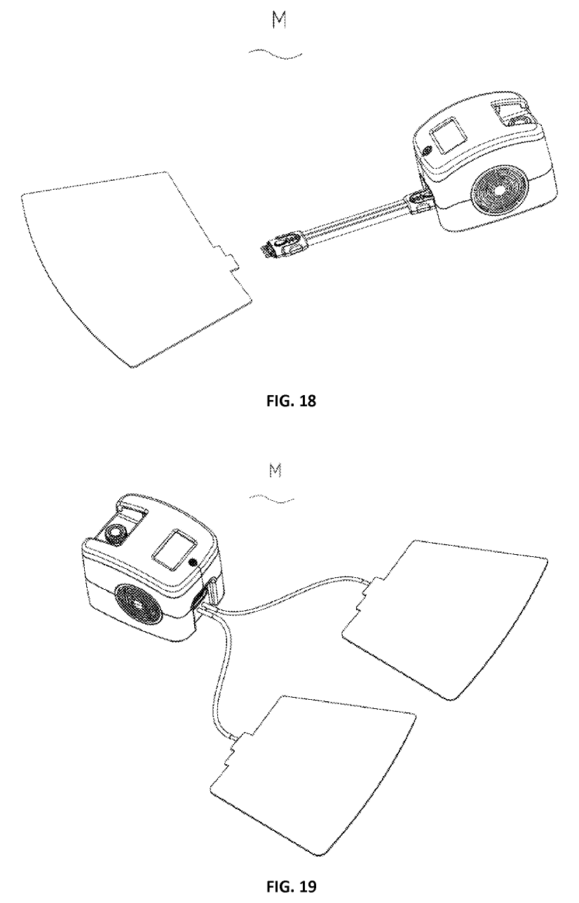

In some examples, referring to FIGS. 1 to 19, the present disclosure provides a portable device M for cold therapy with optional heat and compression therapy including the portable system for cold therapy with optional heat and compression therapy as described in any one of the embodiments as described above. The portable device M for cold therapy with optional heat and compression therapy further includes a first body wrap A, and the first body wrap A is connected to the reservoir 12 through the liquid outlet 122 of the reservoir and the liquid inlet 121 of the reservoir, respectively.

In accordance with the portable device M for cold therapy with optional heat and compression therapy provided in the present disclosure, the advantageous effects include the following. In some examples, the body wrap cooling mechanism is equipped with the compressor 11, the condenser 13, the fan 14, and the heat exchanger 15. Liquid gas is transported from the compressor 11 to the heat exchanger 15 through the first tube 1711. In the heat exchanger 15, the liquid gas undergoes heat exchange with the liquid in the reservoir 12, absorbs heat from the liquid and gasifies, and the liquid temperature decreases. The gasified gas is then transported from the heat exchanger 15 to the condenser 13 through the second tube 172. At this point, the temperature of the gasified gas transported from the heat exchanger 15 to the condenser 13 is relatively high, and the fan 14 is utilized to cool the gasified gas in the condenser 13. The cooled gas is then transported from the condenser 13 to the compressor 11 through the third tube 173, and the compressor 11 compresses and liquefies the gas. This process circulates to cool the liquid in the reservoir 12. A cooling rate is such that the liquid decreases from room temperature to 5°C within about 10 minutes. A cooling power is about 75 watts, and the achieved cooling capacity is about 150 watts. The system exhibits excellent cooling effects, high efficiency, stability, and low power consumption. The liquid with decreased temperature flows into the first body wrap A through the liquid inlet of the first body wrap, providing the body wrap with a cold compression function. In some examples, the air pumps and the solenoid valves are provided to inflate the body wrap, allowing the body wrap to conform more closely to the therapy area. This enhances the therapy effect of the cold therapy with optional heat and compression therapy. Additionally, the combination of inflation and deflation allows the body wrap to provide medical effects such as massage and compression. In some examples, the compressor 11, condenser 13, and fan 14 are paused by the control assembly. The heating tube 16 is activated by the control assembly to increase the temperature of the liquid in the reservoir 12. A heating rate is such that the liquid reaches 43°C from room temperature within about 6 minutes. The heating power is about 150 watts, and the achieved heating capacity is about 150 watts. The device exhibits excellent heating effects, high efficiency, and stability. The liquid with increased temperature flows into the first body wrap A through the liquid inlet of the first body wrap, providing the body wrap with a hot compression function. The cooling and heating mechanisms are simultaneously incorporated into the portable device M for cold therapy with optional heat and compression therapy provided in the present disclosure. The overall volume of the device is approximately 0.014 m³, with a mass of about 3.8 kg, which has a compact, small, and lightweight structure, facilitating convenient portability. The device is versatile, offering capabilities for cold compression, hot compression, and/or massage, enhancing the user experience.

Furthermore, as the cooling and heating mechanisms in the present disclosure is placed inside or underneath the reservoir 12, which are able to directly or indirectly contact the liquid in the reservoir. This allows pre-cooling or pre-heating of the liquid, thereby enabling immediate use when the user requires therapy.

In one embodiment, the portable device M for cold therapy with optional heat and compression therapy further includes a second body wrap B and a third body wrap C. The second air pump 24 is connected to the second body wrap B through the second air tube 175 and to the third body wrap C through the third air tube 176.

It should be noted that, referring to FIG. 19, when a user only needs the air pressure function, the second body wrap B is able to be connected to the second air pump 24 through the second air tube 175 and the third body wrap C is able to be connected to the second air pump 24 through the third air tube 176. The combination of inflation and deflation allows the second body wrap B and the third body wrap C to provide medical effects such as massage and compression.

In one embodiment, shunt tubes are provided in the first body wrap A.

The description of the embodiments described above enables those skilled in the art to implement or use the present disclosure. Various modifications to these embodiments will be apparent to those skilled in the art, and general principles defined herein may be implemented in other embodiments without departing from the spirit or scope of the present disclosure. Therefore, the present disclosure is not limited to the embodiments described herein but is to be accorded the widest scope consistent with the principles and novel features disclosed herein.

What is claimed is:

1. An encryption connection system, comprising:
an accessory module, disposed on a body wrap and configured to store an identity information;
a processing module, connected to the accessory module and configured to obtain the identity information; and
a host module, disposed in a cold therapy system and connected to the processing module, configured to obtain the identity information through the processing module and perform verification and matching,
wherein the host module controls an operating state of the cold therapy system according to a result of the verification and matching.

2. The encryption connection system according to claim 1, wherein the accessory module comprises an authentication carrier; the identity information comprises an identification information, and the identification information is stored in the authentication carrier; and
the host module comprises a main control board; the main control board comprises an identification unit, and the identification unit is configured to read the identification information to complete identification.

3. The encryption connection system according to claim 2, wherein the identity information further comprises a first authentication information, and the first authentication information is stored in the authentication carrier; and
the main control board further comprises an encryption algorithm unit and a comparison unit; the encryption algorithm unit is configured to obtain the identification information and generate a second authentication information; the comparison unit is configured to compare the first authentication information and the second authentication information to complete a match.

4. The encryption connection system according to claim 3, wherein the main control board further comprises a counting unit; the counting unit is configured to record the usage count corresponding to each identification information.

5. The encryption connection system according to claim 3, wherein the processing module comprises a circuit board, and the circuit board is electrically connected to the main control board; and
the circuit board comprises an information reading and writing unit and a signal antenna;
wherein the information reading and writing unit is communicatively connected to the authentication carrier through the signal antenna; the information reading and writing unit is configured to write the first authentication information into the authentication carrier, and to read the identity information from the authentication carrier.

6. The encryption connection system according to claim 5, wherein the processing module further comprises an encryption pipeline assembly, configured to connect the cold therapy system and the body wrap; the encryption pipeline assembly comprises an encryption pipeline, a first connector, and a second connector, wherein the first connector and the second connector are respectively arranged at two ends of the encryption pipeline; and
the accessory module further comprises a third connector; the host module further comprises a fourth connector; the encryption pipeline assembly is detachably connected to the body wrap via the first connector and the third connector; the encryption pipeline assembly is detachably connected to the cold therapy system via the second connector and the fourth connector.

7. The encryption connection system according to claim 6, wherein the information reading and writing unit and the signal antenna are both arranged inside or on a surface of the first connector; the authentication carrier is arranged inside or on a surface of the third connector; and
the encryption pipeline assembly further comprises a power line and a first signal line, wherein both the power line and the first signal line are embedded in a pipe wall of the encryption pipeline; the main control board is electrically connected to the circuit board via the power line and the first signal line; and
when the first connector is mated with the third connector, the signal antenna and the authentication carrier are spatially aligned to establish a near-field communication connection, enabling the information reading and writing unit to acquire the identity information stored in the authentication carrier.

8. The encryption connection system according to claim 7, wherein the power line and the first signal line pass through the second connector and protrude to form a first protrusion; the fourth connector is provided with a first pin member; the main control board is electrically connected to the first protrusion through the first pin member.

9. The encryption connection system according to claim 6, wherein the information reading and writing unit is arranged inside the cold therapy system; the signal antenna is arranged inside or on a surface of the first connector; the authentication carrier is arranged inside or on a surface of the third connector; and
the encryption pipeline assembly further comprises a second signal line, wherein the second signal line is embedded in a pipe wall of the encryption pipeline; the information reading and writing unit is electrically connected to the signal antenna through the second signal line;
when the first connector is mated with the third connector, the signal antenna and the authentication carrier are spatially aligned to establish a near-field communication connection, enabling the information reading and writing unit to acquire the identity information stored in the authentication carrier.

10. The encryption connection system according to claim 9, wherein the second signal line passes through the second connector and protrudes to form a second protrusion; the fourth connector is provided with a second pin member; the information reading and writing unit is electrically connected to the second protrusion through the second pin member.

11. The encryption connection system according to claim 6, wherein the information reading and writing unit and the signal antenna are both arranged inside the cold therapy system; the authentication carrier is arranged inside or on a surface of the third connector;
when the third connector approaches the cold therapy system within a preset sensing distance range, the signal antenna and the authentication carrier establish a far-field communication connection through a spatial electromagnetic field, enabling the information reading and writing unit to acquire the identity information stored in the authentication carrier.

12. The encryption connection system according to claim 3, wherein the processing module comprises a first wireless unit, and the first wireless unit is electrically connected to the main control board; and
the authentication carrier comprises a second wireless unit; the first wireless unit and the second wireless unit are connected via wireless communication; the first wireless unit is configured to write the first authentication information into the second wireless unit, and to read the identity information from the second wireless unit.

13. The encryption connection system according to claim 12, wherein the authentication carrier further comprises a power supply unit; the power supply unit is electrically connected to the second wireless unit and configured to supply operating power to the second wireless unit; and
wherein the power supply unit is a rechargeable battery, a disposable battery, or a charging receiver board.

14. An encryption connection method, comprising:
initiating an identity authentication request from a host module of a cold therapy system to a processing module;
the processing module, according to the identity authentication request, acquiring identity information stored in an accessory module of a body wrap;
the host module receiving the identity information returned by the processing module, and performing verification and matching on the identity information based on a preset verification rule; and
the host module generating a control instruction according to a verification and matching result to control an operating state of the cold therapy system,
wherein, when the verification and matching is successful, the host module authorizes the cold therapy system to start operation; when the verification and matching fails, the host module maintains the cold therapy system in a locked or off state.

15. The encryption connection method according to claim 14, wherein the identity information comprises an identification information and a first authentication information; and
the step of verification and matching comprises:
an identification unit of the host module reading the identification information stored in an authentication carrier of the accessory module to complete identification;
an encryption algorithm unit of the host module generating a second authentication information based on the identification information; and
a comparison unit of the host module comparing the first authentication information and the second authentication information to complete authentication based on a comparison result.

16. The encryption connection method according to claim 14, wherein a communication method between the processing module and the accessory module comprises wireless communication connection or wired electrical connection; and
wherein the wireless communication connection comprises short-range wireless communication based on inductive coupling, including NFC or HF RFID; or long-range wireless communication based on electromagnetic wave radiation, including UHF RFID, Bluetooth, Wi-Fi, ZigBee, or cellular mobile communication.

17. A portable cold therapy device, comprising:
a cold therapy system;
a body wrap; and
an encryption connection system, the encryption connection system comprising:
an accessory module, disposed on the body wrap and configured to store an identity information;
a processing module, connected to the accessory module and configured to acquire the identity information; and
a host module, disposed in the cold therapy system and connected to the processing module, configured to acquire the identity information through the processing module and perform verification and matching,
wherein the host module controls an operating state of the cold therapy system according to a result of the verification and matching.

18. The portable cold therapy device according to claim 17, wherein the cold therapy system comprises a cooling mechanism and a reservoir; the cooling mechanism comprises a cooling component and a first heat exchanger, and the cooling component is used for cooling the first heat exchanger; the host module is electrically connected to the cooling component; the cooling mechanism is placed outside the reservoir; and wherein:
the reservoir comprises a liquid outlet of the reservoir; the first heat exchanger comprises a liquid inlet of the first heat exchanger, and a liquid outlet of the first heat exchanger; the liquid in the reservoir can flow into the first heat exchanger through the liquid outlet of the reservoir and the liquid inlet of the first heat exchanger;
when the liquid in the reservoir flows into the first heat exchanger first, and then flows into the body wrap through the liquid outlet of the first heat exchanger and the liquid inlet of the body wrap; the liquid in the body wrap can flow into the first heat exchanger first through the liquid outlet of the body wrap and the liquid inlet of the first heat exchanger, and then flow into the body wrap again, or the liquid in the body wrap can flow back to the reservoir through the liquid outlet of the body wrap and the liquid outlet of the reservoir.

19. The portable cold therapy device according to claim 18, wherein the cold therapy system further comprises a heating mechanism; the heating mechanism comprises a heating component and a second heat exchanger, and the heating component is used for heating the second heat exchanger; the host module is electrically connected to the heating component; the heating mechanism is placed outside the reservoir; and wherein:
the second heat exchanger comprises a liquid inlet of the second heat exchanger, and a liquid outlet of the second heat exchanger; the liquid in the reservoir can flow into the second heat exchanger through the liquid outlet of the reservoir and the liquid inlet of the second heat exchanger;
when the liquid in the reservoir flows into the second heat exchanger first, and then flows into the body wrap through the liquid outlet of the second heat exchanger and the liquid inlet of the body wrap; the liquid in the body wrap can flow into the second heat exchanger first through the liquid outlet of the body wrap and the liquid inlet of the second heat exchanger, and then flow into the body wrap again, or the liquid in the body wrap can flow back to the reservoir through the liquid outlet of the body wrap and the liquid outlet of the reservoir.

20. The portable cold therapy device according to claim 17, wherein the processing module comprises an encryption pipeline assembly, the encryption pipeline assembly comprises an encryption pipeline, and the cold therapy system is connected to the body wrap through the encryption pipeline to achieve fluid circulation; and
wherein the encryption pipeline comprises an air tube; the cold therapy system further comprises an air pump and a two-way solenoid valve; wherein, the air pump is connected to the body wrap through the air tube, and the two-way solenoid valve is arranged on the air tube;
the air pump and the two-way solenoid valve are both electrically connected to the host module; and
the body wrap is configured to be inflated by the air pump, and the body wrap is configured to be deflated by the two-way solenoid valve.
